(19) United States
(12) Patent Application Publication
(10) Pub. No.: US 2025/0186549 A1
(43) Pub. Date: Jun. 12, 2025

(54) NOVEL FORMULATION COMPRISING MYELOID-DERIVED GROWTH FACTOR

(71) Applicant: BOEHRINGER INGELHEIM INTERNATIONAL GMBH, Ingelheim am Rhein (DE)

(72) Inventors: Jon Michael REED, Ridgefield, CT (US); Cornelia WALTHER, Ingelheim am Rhein (DE); Matthias BERKEMEYER, Ingelheim am Rhein (DE); Priyanka GUPTA, Ridgefield, CT (US); Anton PEKEC, Ingelheim am Rhein (DE); Fabian Alexander MOLL, Basel (CH); Ahmad SEDIQ, Basel (CH)

(21) Appl. No.: 18/972,060

(22) Filed: Dec. 6, 2024

Related U.S. Application Data
(60) Provisional application No. 63/606,920, filed on Dec. 6, 2023.
(60) Provisional application No. 63/607,394, filed on Dec. 7, 2023.

(51) Int. Cl. A61K 38/18; A61K 9/08; A61K 47/18; A61K 47/22; A61K 47/26

(52) U.S. Cl. CPC A61K 38/18; A61K 9/08; A61K 47/183; A61K 47/22; A61K 47/26

(57) ABSTRACT

The present invention generally relates to pharmaceutical formulations which are useful for treating acute coronary syndrome. More specifically, the invention pertains to a novel pharmaceutical formulation which comprises a myeloid-derived growth factor (MYDGF) protein which is useful for treating a myocardial infarction in a subject in need thereof. The pharmaceutical formulation of the invention provides a significantly improved long-term stability compared to commonly used formulations. In addition, the pharmaceutical formulation of the invention allows an easy lyophilisation and reconstitution without any significant loss of the therapeutically active protein. The invention also pertains to a formulation for use in a method for treating a myocardial infarction in a subject in need thereof. The method comprises an intravenous administration of a MYDGF protein to the subject on top of standard of care.

### CROSS-REFERENCE TO RELATED APPLICATIONS

The present application claims the priority benefit of U.S. Provisional Patent Application Ser. No. 63/606,920, filed Dec. 6, 2023, and U.S. Provisional Patent Application Ser. No. 63/607,394, filed Dec. 7, 2023, both entitled NOVEL FORMULATION COMPRISING MYELOID-DERIVED GROWTH FACTOR, and each of which is incorporated by reference in its entirety herein.

### SEQUENCE LISITING

The following application contains a sequence listing submitted electronically as a Standard ST.26 compliant XML file entitled “P 118232_SEQ_LIST.xml,” created on Dec. 4, 2024, as 45,056 bytes in size, the contents of which are incorporated herein.

### BACKGROUND OF THE INVENTION

### Field of the Invention

The present invention generally relates to pharmaceutical formulations which are useful for treating acute coronary syndrome.

### Description of Related Art

Acute myocardial infarction (MI) still is one of the major causes for morbidity and mortality worldwide and a significant contributor to disability. Acute MI is mediated by a thrombotic occlusion of a coronary artery, which leads to progressive cell death in the non-perfused tissue. This triggers an inflammatory response, which leads to scar formation and loss of viable tissue.

Severe alteration of tissue architecture in the left ventricle can cause chamber dilatation, contractile dysfunction and heart failure.

Based on the presence or absence of persistent ST-segment elevation on the electrocardiogram (ECG), ST-segment elevation myocardial infarction (STEMI) can be distinguished from non-ST-segment elevation myocardial infarction (NSTEMI). Both STEMI and NSTEMI are caused by the same pathophysiology and are normally caused by acute thrombosis on a coronary atherosclerotic plaque. In the case of STEMI this often results in total occlusion of the respective coronary artery.

The angiographic pattern for NSTEMI can be more heterogeneous and often point to a critical stenosis rather than a total occlusion of the vessel (Mitsis et al, 2021). The ECG-based separation of these two subsets of patients is presently used for selecting patients who benefit most from immediate thrombolytic reperfusion and primary percutaneous coronary intervention (PCI).

In an emergency situation, PCI leading to rapid reperfusion is the best practice for patients with acute STEMI along with the use of antiplatelet therapy to prevent reocclusion of the coronary artery. Reperfusion is undoubtedly beneficial, evidenced by the decrease of MI-related in-hospital morbidity and mortality rates during the last decades. However, the mortality rate associated with STEMI has plateaued over the last decade and up to 28% of patients develop heart failure (HF) within 90 days post-STEMI despite PCI due to myocardial muscle loss and scarring (Desta et al. 2015). The loss of cardiac tissue and subsequent cardiac remodeling, particularly after a larger myocardial infarction (MI) can induce profound alterations of the left ventricle (LV) tissue architecture leading to chamber dilatation, contractile dysfunction, and ultimately heart failure.

The long-term prognosis also remains poor with more than 50% of first time STEMI patients over 45 years of age develop heart failure or die within a 5-year period (Desta et al, 2017). Whilst PCI can reduce the degree of myocardial scarring in STEMI, it is also paradoxically associated with a process called ‘ischaemia reperfusion injury’ (IRI) through which the myocardium can suffer additional injury at the point of reperfusion due to a proinflammatory “burst”, leading to apoptosis of the cardiomyocytes downstream of the previous occlusion.

Thus, even in the setting of a successful PCI, STEMI patients experience further myocardial damage and scarring. Furthermore, especially after extensive MIs, natural repair mechanisms may be insufficient to prevent adverse remodeling and further scarring. STEMI thus remains a life-threatening emergency indication with a poor prognosis. To date no treatment exists that can be administered in combination with PCI to reduce ischaemic damage and/or reperfusion-induced myocardial injury.

The myeloid-derived growth factor (MYDGF) is a protein that has shown to improve tissue repair and heart function in rodent models of MI (WO 2014/111458). It was found that treatment with recombinant MYDGF is able to protect cardiomyocytes from cell deaths and repair the heart after acute MI. The development of a protein-based therapy would be a promising approach for cardiac repair and potentially also for ischemic repair in other tissues (Ebenhoch et al., 2019; Polten et al., 2019; Botnov et al., 2018, Korf-Klingebiel et al., 2015).

It has been shown that murine MYDGF blocks apoptosis of cardiomyocytes, induced by simulated ischaemia reperfusion. MYDGF signalling is triggered via a yet unknown molecular target/receptor on rat cardiomyocytes and is believed to include a phosphatidylinositol 3-kinase (PI3K) dependent pathway. This results in the phosphorylation of AKT1, which phosphorylates its down-stream targets Bad and Bax. This reduces cytosolic cytochrome c release and caspase-9 cleavage. This decreases the activity of caspase-3 and caspase-7, which are well known effector caspases driving apoptosis (Korf-Klingebiel 2015). In addition, murine MYDGF enhances angiogenesis in vitro by activating mitogen-activated protein kinase (MAPK) pathways in human endothelial cells. This results in phosphorylation of STAT3 which in turn increases cyclin D1 expression ((Korf-Klingebiel 2015). Treatment of mice with MYDGF following myocardial infraction reduced infract size and scar size, increased re-capillarization in the border zone of the infract, improved cardiac function sub-acutely and chronically as assessed by echocardiography, and reduced heart failure associated mortality.

According to studies in the mouse (Korf-Klingebiel et al., 2015), the dosing regimen to achieve an effect in the mouse model of myocardial infarction (ischemia/reperfusion) is a 10 μg bolus injection into the left ventricle cavity of the heart shortly before reperfusion, followed by a 7 day subcutaneous infusion via a minipump with the dose of 10 μg/day. No formulation that allows for prolonged storage has been developed so far.

MYDGF is a large and complex molecule and is subject to various degradation processes, especially in liquid state. The process of producing the protein and the purification needs to be well controlled. Stabilization of the molecule by developing an appropriate formulation is a major challenge. As proteins have various degradation pathways this can result in losing the therapeutic activity.

### SUMMARY OF THE INVENTION

Own studies suggest that a (single) intravenous administration of a myeloid-derived growth factor (MYDGF) protein to the subject on top of standard of care is a suitable to treat (acute) myocardial infarction in a subject, preferably a human subject.

It is an object of the invention to provide a formulation for intravenous injection with or without further dilution. In the formulations provided by the invention, the stability of the MYDGF protein is increased due to stabilizing excipients in the formulation. The stabilization enables the use of MYDGF for intravenous administration in clinical settings.

The MYDGF formulations of the invention have the particular advantage that the liquid formulation can be lyophilized without any adaptation of the excipient amounts or any addition of additional excipients. The option to freeze dry the formulation of the invention provide for extended shelf-life conditions compared to the liquid formulations. For example, the freeze-dried formulations can be stored at ambient temperature for long terms without any significant degradation of the MYDGF protein.

Where the formulation of the invention is in liquid form, it should allow a concentration of 40-60 mg/ml MYDGF and be suitable for intravenous administration. The liquid composition that forms the basis for the formulation should be capable of being lyophilised, stored, and reconstituted before use.

Accordingly, the present invention pertains to a new pharmaceutical formulation which has been optimized for maintaining the stability of a MYDGF protein upon long-term storage of the pharmaceutical formulation and reducing protein degradation.

In a first aspect, the present invention relates to a pharmaceutical formulation comprising:

(a) 0.5 mg/ml to 200 mg/ml of a myeloid-derived growth factor (MYDGF) protein;

(b) 10 mM to 100 mM of a buffer;

(c) 1 mM to 50 mM of a stabilizer;

(d) 20 mM to 250 mM of a tonicity agent; and

(e) 0.01% to 0.1% (w/v) of a surfactant;

wherein the composition has a pH of 5.0 to 7.0.

The pharmaceutical formulation of the invention comprises 5 components, namely a MYDGF protein, a buffering agent, a stabilizer, tonicity agent and a surfactant.

As a first component, the formulation comprises a buffering agent. According to the invention the buffering agent is an acetate buffer, a citrate buffer, a histidine buffer, a succinate buffer, a phosphate buffer, or a tromethamine buffer.

The concentration of the buffer which is present in the pharmaceutical formulation of the invention is 10 mM to 100 mM. Preferably, the concentration of the buffer in the formulation is 10 mM to 75 mM, 10 mM to 50 mM, and more preferably 10 mM to 30 mM. Most preferably, the concentration of the buffer in the formulation is 20 mM.

It is preferred that the buffer in the pharmaceutical formulation of the invention is a histidine buffer. The histidine buffer can be a L-histidine/HCl buffer, i.e. a buffer which comprises L-histidine or mixtures of L-histidine and L-histidine hydrochloride. The pH of the histidine buffer can be adjusted with hydrochloric acid.

Thus, it is particularly preferred that the pharmaceutical formulation of the invention comprises a histidine buffer which is present in the formulation at a concentration of 10 mM to 100 mM. Preferably, the concentration of the histidine buffer in the formulation is 10 mM to 75 mM, 10 mM to 50 mM, and more preferably 10 mM to 30 mM. Most preferably, the concentration of the histidine buffer in the formulation is 20 mM.

As a second component, the formulation comprises a stabilizer. According to the invention the stabilizer is an amino acid, such as methionine, arginine, glycine, proline, lysine, or cysteine.

The concentration of the stabilizer which is present in the pharmaceutical formulation of the invention is 1 mM to 50 mM. Preferably, the concentration of the stabilizer in the formulation is 1mM to 40 mM, 1 mM to 30 mM, 1 mM to 20 mM or 1 mM to 10 mM. In some embodiments, the concentration of the stabilizer in the formulation is 2 mM to 15 mM, 5 mM to 15 mM, and more preferably 5 mM to 10 mM. Most preferably, the concentration of the stabilizer in the formulation is 10 mM.

It is preferred that the stabilizer in the pharmaceutical formulation of the invention is methionine.

Thus, it is particularly preferred that the pharmaceutical formulation of the invention comprises methionine which is present in the formulation at a concentration of 1 mM to 20 mM. Preferably, the concentration of the methionine in the formulation is 2 mM to 15 mM, 5 mM to 15 mM, and more preferably 5 mM to 10 mM. Most preferably, the concentration of the methionine in the formulation is 10 mM.

As a third component, the formulation comprises a tonicity agent. According to the invention the tonicity agent is a sugar or sugar alcohol, such as sucrose, trehalose, sorbitol, mannitol, or dextrose. The concentration of the tonicity agent which is present in the pharmaceutical formulation of the invention is 20 mM to 250 mM. Preferably, the concentration of the tonicity agent in the formulation is 50 mM to 250 mM, 100 mM to 250 mM, 150 mM to 250 mM, 175 mM to 250 mM, and more preferably 200 mM to 250 mM, such as 200 mM to 240 mM. Most preferably, the concentration of the tonicity agent in the formulation is 220 mM.

It is preferred that the tonicity agent which is included in the pharmaceutical formulation of the invention is sucrose.

Thus, it is particularly preferred that the pharmaceutical formulation of the invention comprises sucrose which is present in the formulation at a concentration of 20 mM to 250 mM. Preferably, the concentration of sucrose in the formulation is 50 mM to 250 mM, 100 mM to 250 mM, 150 mM to 250 mM, 175 mM to 250 mM, and more preferably 200 mM to 250 mM, such as 200 mM to 240 mM. Most preferably, the concentration of sucrose in the formulation is 220 mM.

As a fourth component, the formulation comprises a surfactant.

As used herein, the term “surfactant” refers to a surface-active agent. Examples of pharmaceutically acceptable surfactants include polyoxyethylen-sorbitan fatty acid esters (Tween), poly-oxyethylene alkyl ethers (e.g., Brij), alkylphenylpolyoxyethylene ethers (e.g., Triton X), polyoxyethylene-polyoxypropylene copolymers (e.g., Poloxamer, Pluronic), and sodium dodecyl sulphate (SDS). The surfactant preferably is a polyoxyethylen-sorbitan fatty acid ester, i.e. a polysorbate, or a non-ionic polyoxyethylene-polyoxypropylene copolymer, such as a poloxamer. Preferred polysorbates for use in the formulation of the invention comprise polysorbate 20 and polysorbate 80. A preferred poloxamer for use in the formulation of the invention is Poloxamer 188™. Preferably, a non-ionic surfactant is used.

The concentration of the surfactant which is present in the pharmaceutical formulation of the invention is 0.01% to 0.1% (w/v). Preferably, the concentration of the surfactant in the formulation is 0.02% to 0.1% (w/v), 0.02% to 0.08% (w/v), 0.02% to 0.07% (w/v), 0.02% to 0.06% (w/v), 0.02% to 0.05% (w/v), and more preferably 0.02% to 0.04% (w/v). Most preferably, the concentration of the surfactant in the formulation is 0.03% (w/v) or 0.04% (w/v).

It is preferred that the surfactant which is included in the pharmaceutical formulation of the invention is polysorbate 20.

Thus, it is particularly preferred that the pharmaceutical formulation of the invention comprises polysorbate 20 which is present in the formulation at a concentration of 0.01% to 0.1% (w/v). Preferably, the concentration of polysorbate 20 in the formulation is 0.02% to 0.1% (w/v), 0.02% to 0.08% (w/v), 0.02% to 0.07% (w/v), 0.02% to 0.06% (w/v), 0.02% to 0.05% (w/v), and more preferably 0.02% to 0.04% (w/v). Most preferably, the concentration of polysorbate 20 in the formulation is 0.03% (w/v) or 0.04% (w/v).

In another embodiment, it is preferred that the surfactant which is included in the pharmaceutical formulation of the invention is polysorbate 80.

Thus, it is particularly preferred that the pharmaceutical formulation of the invention comprises polysorbate 80 which is present in the formulation at a concentration of 0.01% to 0.1% (w/v). Preferably, the concentration of polysorbate 80 in the formulation is 0.02% to 0.1% (w/v), 0.02% to 0.08% (w/v), 0.02% to 0.07% (w/v), 0.02% to 0.06% (w/v), 0.02% to 0.05% (w/v), and more preferably 0.02% to 0.04% (w/v). Most preferably, the concentration of polysorbate 80 in the formulation is 0.03% (w/v) or 0.04% (w/v).

The MYDGF protein that is used in the pharmaceutical formulation of the invention preferably comprises or consists of an amino acid sequence selected from the group of different MYDGF proteins which are set forth as SEQ ID NO:1, SEQ ID NO:2, SEQ ID NO:3, SEQ ID NO:6, SEQ ID NO: 11 and SEQ ID NO:13. All of these variants are derived from human MYDGF.

Due to the low amount of anti-drug antibodies (ADA) caused by this protein variant, the use of a MYDGF protein comprising or consisting of the sequence of SEQ ID NO:1 is particularly preferred for use in the pharmaceutical formulation of the invention. In addition, the MYDGF protein of SEQ ID NO:1 can be produced in high amounts and desirable purity. According to the invention, the MYDGF protein is contained in the formulation of the invention at a concentration of 0.5 mg/ml to 200 mg/ml, and preferably at a concentration of 1 mg/ml to 200 mg/ml, 5 mg/ml to 200 mg/ml, 10 mg/ml to 200 mg/ml, or 25 mg/ml to 200 mg/ml. More preferably, the MYDGF protein is contained in the formulation of the invention at a concentration of 1 mg/ml to 100 mg/ml, such as 10 mg/ml to 90 mg/ml, 20 mg/ml to 80 mg/ml, 30 mg/ml to 70 mg/ml, 40 mg/ml to 60 mg/ml. A concentration of 50 mg/ml is particularly preferred.

The pharmaceutical formulation of the invention has a pH of 5.0 to 7.0, and preferably a pH of 5.0 to 6.5, a pH of 5.2 to 6.5, a pH of 5.2 to 6.2, a pH of 5.4 to 6.0. In some embodiments, the pharmaceutical formulation of the invention has a pH of 5.5 to 6.5, a pH of 5.6 to 6.4, a pH of 5.7 to 6.3, a pH of 5.8 to 6.2, a pH of 5.9 to 6.1. A pH of 5.7 +0.3 is particularly preferred according to the invention.

In one embodiment of the invention, the pharmaceutical formulation of the invention comprises or consists of:

(a) 0.5 mg/ml to 200 mg/ml of a myeloid-derived growth factor (MYDGF) protein;

(b) 10 mM to 100 mM of a histidine buffer;

(c) 1 mM to 50 mM methionine;

(d) 20 mM to 250 mM sucrose; and

(e) 0.01% to 0.1% (w/v) polysorbate 20 or polysorbate 80;

wherein the composition has a pH of 5.0 to 7.0.

In one embodiment of the invention, the pharmaceutical formulation of the invention comprises or consists of:

(a) 40 mg/ml to 60 mg/ml of a myeloid-derived growth factor (MYDGF) protein;

(b) 10 mM to 30 mM of a histidine buffer;

(c) 5 mM to 15 mM methionine;

(d) 100 mM to 250 mM sucrose; and (e) 0.02% to 0.06% (w/v) polysorbate 20 or polysorbate 80;

wherein the composition has a pH of 5.0 to 6.5.

In yet another embodiment of the invention, the pharmaceutical formulation of the invention comprises or consists of:

(a) 45 mg/ml to 55 mg/ml of a myeloid-derived growth factor (MYDGF) protein;

(b) 18 mM to 22 mM of a histidine buffer;

(c) 9 mM to 11 mM methionine;

(d) 200 mM to 240 mM sucrose; and

(e) 0.02% to 0.06% (w/v) polysorbate 20;

wherein the composition has a pH of 5.7+0.3.

In yet another embodiment of the invention, the pharmaceutical formulation of the invention comprises or consists of:

(a) 45 mg/ml to 55 mg/ml of a myeloid-derived growth factor (MYDGF) protein;

(b) 18 mM to 22 mM of a histidine buffer;

(c) 9 mM to 11 mM methionine;

(d) 200 mM to 240 mM sucrose; and

(e) 0.02% to 0.06% (w/v) polysorbate 80;

wherein the composition has a pH of 5.7 +0.3.

In yet another embodiment of the invention, the pharmaceutical formulation of the invention comprises or consists of:

(a) 50 mg/ml of a myeloid-derived growth factor (MYDGF) protein;

(b) 20 mM of a histidine buffer;

(c) 10 mM methionine;

(d) 220 mM sucrose; and

(e) 0.03% (w/v) or 0.04% (w/v) polysorbate 20;

wherein the composition has a pH of 5.7+0.3.

In yet another embodiment of the invention, the pharmaceutical formulation of the invention comprises or consists of:

(a) 50 mg/ml of a myeloid-derived growth factor (MYDGF) protein;

(b) 20 mM of a histidine buffer;

(c) 10 mM methionine;

(d) 220 mM sucrose; and

(e) 0.03% (w/v) or 0.04% (w/v) polysorbate 80;

wherein the composition has a pH of 5.7+0.3.

In yet another embodiment of the invention, the pharmaceutical formulation of the invention comprises or consists of:

(a) 50 mg/ml of a myeloid-derived growth factor (MYDGF) protein which comprises the amino acid sequence of SEQ ID NO: 4.

(b) 20 mM of a histidine buffer;

(c) 10 mM methionine;

(d) 220 mM sucrose; and

(e) 0.03% (w/v) or 0.04% (w/v) polysorbate 20 or polysorbate 80;

wherein the composition has a pH of 5.7±0.3.

In yet another embodiment of the invention, the pharmaceutical formulation of the invention comprises or consists of:

(a) 50 mg/ml of a myeloid-derived growth factor (MYDGF) protein which comprises or consists the amino acid sequence of SEQ ID NO:1.

(b) 20 mM of a histidine buffer;

(c) 10 mM methionine;

(d) 220 mM sucrose; and

(e) 0.03% (w/v) or 0.04% (w/v) polysorbate 20;

wherein the composition has a pH of 5.7+0.3.

In yet another embodiment of the invention, the pharmaceutical formulation of the invention comprises or consists of:

(a) 50 mg/ml of a myeloid-derived growth factor (MYDGF) protein which comprises or consists the amino acid sequence of SEQ ID NO:1.

(b) 20 mM of a histidine buffer;

(c) 10 mM methionine;

(d) 220 mM sucrose; and

(e) 0.03% (w/v) or 0.04% (w/v) polysorbate 80;

wherein the composition has a pH of 5.7+0.3.

The pharmaceutical formulation of the invention can be provided either in liquid form or in lyophilized form, i.e. as a dry powder. Such powder can be reconstituted by adding a liquid to the powder, e.g., water for injection (WFI) or bacteriostatic water for injection (BWFI). It is an advantage of the formulation of the invention that no additional excipients have to be added to render the formulation suitable for being lyophilized. Pictures of the freeze dried formulation are included in FIG. 9. It is preferred in one embodiment that the above formulations consist of components (a)-(e) and water, and do not contain any other components and excipients. Due to the rapid reconstitution, a fast administration is guaranteed. Stability of the freeze dried formulation stored at 25° C. for up to 6 months showed no significant degradation of the MYDGF protein. Data is evaluated by monomer content by SEC-HPLC (shown in FIG. 7) and main peak by ion exchange chromatography (shown in FIG. 8).

The pharmaceutical formulation of the invention exerts a particularly preferred storage stability, as described in the below examples and shown in FIGS. 5 and 6, the formulation of the invention was found to be extremely stable when stored at 5° C. for 18 or 24 months. Preferably, the pharmaceutical formulation of the invention shows a degradation of MYDGF protein of less than 10.0%, less than 9.0%, less than 8.0%, less than 7.0%, less than 6.0%, or less than 5.0%, when stored at 5° C. for 18 or 24 months, as determined by ion exchange chromatography or size exclusion chromatography.

In a second aspect, the present invention relates to a pharmaceutical formulation according to the first aspect of the invention for use in medicine.

The pharmaceutical formulation can be administered directly as a bolus injection intravenously or via infusion by dilution with infusion media. Standard infusion medias are, e.g., glucose solution and sodium chloride solution. Examples of dilution with sodium chloride solution without degradation of MYDGF protein is included in FIG. 10, monomer content examined by size exclusion chromatography.

In a third aspect, the present invention relates to a pharmaceutical formulation according to the first aspect of the invention for use in a method of treating a myocardial infarction in a subject. Preferably, the myocardial infarction which is to be treated by the formulation of the invention is a ST-segment elevation myocardial infarction (STEMI). More preferably, the myocardial infarction to be treated is an anterior STEMI, i.e., a STEMI that results from occlusion of a subject's left anterior descending artery (LAD). The myocardial infarction to be treated may be a STEMI that is associated with a cardiogenic shock. The subject to be treated preferably is a mammal, and more preferably a human. The pharmaceutical formulation of the invention can be administered by different routes. However, it is preferred that the formulation of the invention is administered intravenously. Accordingly, the formulation of the invention can be formulated for different routes of administration. It is preferred that the formulation of the invention is formulated for intravenous administration, e.g., by intravenous infusion. The pharmaceutical formulation of the invention preferably is for use in a method of treating a myocardial infarction in a subject, wherein said method further comprises percutaneous coronary intervention (PCI) to restore blood flow to the heart tissue.

In a fourth aspect, the present invention relates to a method of treating a myocardial infarction in a subject, said method comprising the administration of a pharmaceutical formulation according to the first aspect of the invention. Preferably, the myocardial infarction which is to be treated by the method according to the fourth aspect of the invention is a ST-segment elevation myocardial infarction (STEMI). More preferably, the myocardial infarction to be treated is an anterior STEMI, i.e., a STEMI that results from occlusion of a subject's left anterior descending artery (LAD). The myocardial infarction to be treated may be a STEMI that is associated with a cardiogenic shock. The subject to be treated preferably is a mammal, and more preferably a human. The method preferably includes the administration of a pharmaceutical formulation according to the first aspect of the invention by intravenous infusion. The method may further comprise percutaneous coronary intervention (PCI) to restore blood flow to the heart tissue.

Any of the above-recited treatment methods can also be applied in a subject suffering from myocardial infarction for one or more of the following purposes:
reducing the infarct size,
reducing the scar size,
improving cardiac output,
preventing or ameliorating heart failure (HF),
reducing hospitalization for heart failure (HHF),
reducing mortality, and/or
reducing reperfusion injury.

In a particularly preferred embodiment, the above-recited treatment methods are applied for reducing the infarct size as measured by late gadolinium enhancement cardiac magnetic resonance (LGE-CMR) according to Ibanez et al. 2019. More preferably, the above-recited treatment methods are applied for reducing the infarct size as measured by LGE-CMR according to Ibanez et al. 2019 after PCI.

Furthermore, any of the above-recited treatment methods can be combined with commonly known interventions or medicinal products that are directed to the treatment of MI.

### BRIEF DESCRIPTION OF THE FIGURES

FIG. 1 shows the main peak results measured by ion exchange chromatography (IEC-HPLC) (Y-axis) after storage of two weeks at 40°.

FIG. 2 shows the turbidity results (Y-axis) initially and after storage for 9 months at 25° C.

FIG. 3 shows the main peak results measured by size exclusion chromatography (SEC-HPLC) (Y-axis) of formulations during storage at 25° C. for up to 9 months.

FIG. 4 shows the main peak results measured by ion exchange chromatography (IEC-HPLC) (Y-axis) of formulations during storage at 25° C. for up to 9 months.

FIG. 5 shows the main peak measured by size exclusion chromatography (SEC-HPLC) (Y-axis) of formulations during storage at 5° C. for up to 24 months.

FIG. 6 shows the main peak area determined by ion exchange chromatography (IEC-HPLC) (Y-axis) of formulations during storage at 5° C. for up to 24 months.

FIG. 7 shows the main peak measured by size exclusion chromatography (SEC-HPLC) (Y-axis) of lyophilized Formulation H during storage at 25° C. for up to 6 months.

FIG. 8 shows the main peak measured by ion exchange chromatography (IEC-HPLC) (Y-axis) of lyophilized Formulation H during storage at 25° C. for up to 6 months.

FIG. 9 shows pictures and μCT scans of formulations after freeze drying.

FIG. 10 shows the main peak measured by size exclusion chromatography (SEC-HPLC) (Y-axis) of solutions non diluted and diluted prior and after storage at 25° C.

FIG. 11 shows the turbidity results (Y-axis) of solutions non diluted and diluted prior and after storage at 25° C.

### EXAMPLES

The invention will be illustrated by the following Examples which are given by way of example only. Specifically, Examples 1-3 describe the generation of the production strain and the heterologous expression and purification of MYDGF variants. The other Examples describe studies which are directed to the stability of MYDGF formulations.

### Example 1: Production of Recombinant Human MYDGF Proteins

The preparation of MYDGF variants as well as functional assays for testing their therapeutic efficiency are also described in WO 2023/233034 A1. The following MYDGF proteins were prepared and used in the below studies and experiments:

1.1 Human [+G] MYDGF variant HEK (“h[+G]-MYDGF-HEK” hereinafter) h[+G]-MYDGF-HEK has the sequence SEQ ID NO: 11 (amino acid sequence of the [+G] MYDGF variant, in which the N-terminal V residue in position +1 of the mature human MYDGF is preceded by an G residue):

h[+G]-MYDGF-HEK has the sequence
SEQ ID NO: 11
GVSEPTTVAFDVRPGGVVHSFSHNVGPGDKYTCMFTYASQGGTNEQWQM
SLGTSEDHQHFTCTIWRPQGKSYLYFTQFKAEVRGAEIEYAMAYSKAAF
ERESDVPLKTEEFEVTKTAVAHRPGAFKAELSKLVIVAKASRTEL
The h[+G]-MYDGF-HEK precursor has the sequence
SEQ ID NO: 12
MGWSLILLFLVAVATRVLSHHHHHHAGSENLYFQ↓GVSEPTTVAFDVRP
GGVVHSFSHNVGPGDKYTCMFTYASQGGTNEQWQMSLGTSEDHQHFTCT
IWRPQGKSYLYFTQFKAEVRGAEIEYAMAYSKAAFERESDVPLKTEEFE
VTKTAVAHRPGAFKAELSKLVIVAKASRTEL

The variant was manufactured as described in Polten, F. et al. (2019), Anal Chem, 91, 1302-1308 on page 1303, 1st column and Figure S1, and in Ebenhoch, R. et al. (2019), Nat Commun 10, 5379 on page 8, left column. The precursor sequence of SEQ ID NO: 12 has a His-tag integrated at the N-Terminus that is cleaved by TEV protease so that the protein according to Seq ID NO: 11 is formed.

1.2 Human MYDGF Variant with HIS Tag (“hMYDGF-[HIS]-HEK” Hereinafter)

hMYDGF-[HIS]-HEK has the sequence
SEQ ID NO: 13
VSEPTTVAFDVRPGGVVHSFSHNVGPGDKYTCMFTYASQGGTNEQWQMS
LGTSEDHQHFTCTIWRPQGKSYLYFTQFKAEVRGAEIEYAMAYSKAAFE
RESDVPLKTEEFEVTKTAVAHRPGAFKAELSKLVIVAKASRTELHHHHH
H
The hMYDGF-[HIS]-HEK precursor has the sequence
SEQ ID NO: 14
MGWSLILLFLVAVATRVLSIVSEPTTVAFDVRPGGVVHSFSHNVGPGDK
YTCMFTYASQGGTNEQWQMSLGTSEDHQHFTCTIWRPQGKSYLYFTQFK
AEVRGAEIEYAMAYSKAAFERESDVPLKTEEFEVTKTAVAHRPGAFKAE
LSKLVIVAKASRTELHHHHHH

It is produced using a stable HEK293f (human embryonic kidney) cell line.

Culture media: F17 supplemented with 0.1% Pluronic F-68, 6 mM GlutaMAX

Culture maintenance: Five vials of cells were cryopreserved previously for quotation 1409 and stored in liquid N2. For the current quotation, one vial of cells was thawed and placed into 5 mL of pre-warmed media. The cells were then centrifuged at 300×g for 5 min, resuspended at a density of 5×105 cells/mL, and transferred to a T-75 flask for static culture. The following day, cells were transferred to a shake flask and incubated at 37° C. in a humidified 5% CO2 environment with shaking at 135 rpm. The MYDGF-His stable HEK293f cells were maintained antibiotic-free at a density between 0.5-4×106 cells/mL in shake flasks. The flasks were incubated at 37° C. in a humidified 5% CO2 environment with shaking at 135 rpm.

Culture volume: 40 L (40×1 L in 2 L shake flasks); Initial density: 0.5×106 cells/mL; Method: Forty, 1 L cultures of MYDGF-His stable HEK293f cells in 2 L shake flasks were seeded at a density of 0.5×106 cells/mL. Culture parameters were monitored using a ViCell XR for density and viability. Density, viability, and average diameter measurements were performed on a Vi-Cell XR.; Harvest: Culture was harvested 10 days post transfection via centrifugation at 4° C. for 5 minutes at 1000×g. The conditioned culture supernatant (CCS) was clarified by centrifugation at 4° C. for 30 minutes at 9300×g. For expression analysis, the cells were harvested from 1 mL of culture by centrifugation for 5 minutes at 1000×g (25° C.). The cell pellet was lysed via freeze/thaw and resuspended in 1× TBS pH 8.0, 0.1% BOG, 0.1% DDM, 1 mM βME, 10 U/mL Turbonuclease, 1× Complete® protease inhibitor cocktail (10 μL per 1×105 cells). Lysis was verified by light microscopy.

1.3 Human [+A] MYDGF Variant E. coli (“h[+A]-MYDGF-E. coli” Hereinafter”)

h[+A]-MYDGF-E.coli corresponds to the [+A] variant described below and has a sequence set forth in SEQ ID NO:1.

This protein can be manufactured as follows:

### 1.3.1 Preparation of Vectors and Transfection

For the production of a cell bank, a derivative of Escherichia coli strain BL21 (DE3) was used that had been modified such that it does not produce phages. The strain was transformed with one of the vectors set forth in SEQ ID NO:7-10 carrying the gene encoding the respective MYDGF variant. The genes of the respective variants were codon-optimized for high expression rates in E. coli and synthesized by ATUM (Newark, California, USA). Plasmids encoding the following MYDGF variants were produced:
[+A] variant (h[+A]-MYDGF-E coli) in which the N-terminal V residue in position +1 of the mature human MYDGF is preceded by an A residue (aa sequence set forth in SEQ ID NO: 1: AVSEPTTVAFDVRPGGVVHSFSHNVGPGDKYTCMFTYASQGGTNEQWQMSLGT SEDHQHFTCTIWRPQGKSYLYFTQFKAEVRGAEIEYAMAYSKAAFERESDVPLK TEEFEVTKTAVAHRPGAFKAELSKLVIVAKASRTEL). A possible expression vector encoding this variant is set forth in SEQ ID NO:7.
[+S] variant (h[+S]-MYDGF-E coli) in which the N-terminal V residue in position +1 of the mature human MYDGF is preceded by an S residue (aa sequence set forth in SEQ ID NO: 2: SVSEPTTVAFDVRPGGVVHSFSHNVGPGDKYTCMFTYASQGGTNEQWQMSLGT SEDHQHFTCTIWRPQGKSYLYFTQFKAEVRGAEIEYAMAYSKAAFERESDVPLK TEEFEVTKTAVAHRPGAFKAELSKLVIVAKASRTEL). A possible expression vector encoding this variant is set forth in SEQ ID NO:8.
[+G] variant (h[+G]-MYDGF-E coli) in which the N-terminal V residue in position +1 of the mature human MYDGF is preceded by an G residue (aa sequence set forth in SEQ ID NO: 3: GVSEPTTVAFDVRPGGVVHSFSHNVGPGDKYTCMFTYASQGGTNEQWQMSLGT SEDHQHFTCTIWRPQGKSYLYFTQFKAEVRGAEIEYAMAYSKAAFERESDVPLK TEEFEVTKTAVAHRPGAFKAELSKLVIVAKASRTEL. A possible expression vector encoding this variant is set forth in SEQ ID NO:9.
[−V] variant (h[−V]-MYDGF-E coli) in which the N-terminal V residue in position +1 of the mature human MYDGF is lacking (aa sequence set forth in SEQ ID NO:4: SEPTTVAFDVRPGGVVHSFSHNVGPGDKYTCMFTYASQGGTNEQWQMSLGTSE DHQHFTCTIWRPQGKSYLYFTQFKAEVRGAEIEYAMAYSKAAFERESDVPLKTE EFEVTKTAVAHRPGAFKAELSKLVIVAKASRTEL. A possible expression vector encoding this variant is set forth in SEQ ID NO:10.

In case of a discrepancy between any of the sequences listed above and the sequences set forth in the attached sequence listing, the sequence listing will prevail. Hyphens that eventually show up within the sequence are a result of truncation due to text processing and must be ignored.

To prepare the expression strain, E. coli cells were transformed with the above described vector plasmids by electroporation using a Gene Pulser Xcell™ Electroporation System (BioRad). The protein was expressed in E. coli cell in the form of inclusion bodies (IBs) that accumulated in the cytoplasm, as further described below.

1.3.2 Expression of MYDGF Variants

The expression and purification of the MYDGF variants was performed by the following general scheme:
1. Fermentation
2. IB Preparation
3. Solubilization/Refolding
4. Ultrafiltration/Diafiltration
5. Anion exchange chromatography
6. Preferably: Hydrophobic Interaction Chromatography
7. Preferably: Concentration and Formulation

### Fermentation

One cell bank vial of the production strain obtained from was thawed at room temperature. A pre-culture (PC) consisted of two 1 L shake flasks with 300 mL seed culture medium per flask. The composition of the seed medium is depicted in below Table 1. All buffer and media were prepared with reverse osmosis (RO) water and sterilized before use using either nanofiltration devices or heat-sterilization.

TABLE 1
Seed Culture Medium

| | Amount | |
|---|---|---|
| Component | per liter | Unit |

| Solution A (Basic Medium) | | | |
|---|---|---|---|
| 1 | Potassium Dihydrogen Phosphate | 3 | g |
| 2 | Di-Potassium Hydrogen Phosphate | 4.58 | g |
| 3 | Bacto Yeast Extract | 0.5 | g |
| 4 | Sodium Citrate × 2H₂O | 1 | g |
| 5 | Trace Element Solution | 0.2 | mL |
| | | 0.22 | g |
| 6 | Magnesium Sulfate × 7 H₂O | 0.4 | g |
| 7 | Ammonium Sulfate | 1.8 | g |
| 8 | Ammonium Chloride | 1.48 | g |
| 9 | RO-Water, Ad | 900 | mL |
| | | 906.93 | g |
| 10 | Antifoam (if needed) | | mL |
| Solution B (Sterile Additions) | | | |
| 11 | Calcium Chloride 2H₂O | 0.08 | g |
| 12 | Glucose anhydrous | 10 | g |
| 13 | RO-Water, ad | 100 | mL |
| | | 103.74 | g |
| 14 | Antibiotic (if needed) | | mL |

Antifoam and antibiotic were added as needed. Kanamycin was added as an antibiotic (to a final concentration of 50 μg/mL). Each shake flask was inoculated with 100 μL of the production strain. The cultures were grown for approximately 9.4 h, aiming for an OD (optical density at 550 nm) of 1.75±0.5.

The main culture (MC) was performed in a 20 L gross stainless-steel bioreactor that contained 10 L batch medium. The composition of the batch medium is depicted in below Table 2.

TABLE 2
Batch Medium

| | Amount | |
|---|---|---|
| Component | per liter | Unit |

| Solution A (Basic Medium) | | | |
|---|---|---|---|
| 1 | Potassium Dihydrogen Phosphate | 3 | g |
| 2 | Di-Potassium Hydrogen Phosphate | 4.58 | g |
| 3 | Sodium Citrate × 2 H₂O | 1 | g |
| 4 | Trace Element Solution | 0.2 | mL |
| | | 0.22 | g |
| 5 | Magnesium Sulfate × 7 H₂O | 0.4 | g |
| 6 | Ammonium Sulfate | 1.8 | g |
| 7 | Ammonium Chloride | 1.48 | g |
| 8 | RO-Water, ad. | 900 | mL |
| | | 906.93 | g |
| 9 | Polypropylene glycol 2000 | 1 | mL |
| | | 1 | g |
| Solution B (Sterile Additions) | | | |
| 10 | Calcium Chloride × 2 H₂O | 0.08 | g |
| 11 | Glucose anhydrous | 10 | g |
| 12 | RO-Water, ad. | 100 | mL |
| | | 103.74 | g |

The batch medium was inoculated with 100 mL cell broth from the pre-culture. In the batch phase and exponential feed phase, fermentation process parameters were held constant at 33.5° C., pH 6.8, 1.0 bar head pressure and a DO set-point of 20%. An exponential feed (concentration 600 g/L glucose; μ=0.25 h-1) was started after carbon source depletion was observed via a dissolved oxygen (DO) peak. The composition of the feed medium is depicted in below Table 3.

TABLE 3
Feed Medium

| | | Amount | |
|---|---|---|---|
| Component | | per liter | Unit |
| Solution A (Basic Medium) | | | |
| 1 | Glucose anhydrous | 600 | g |
| 2 | Calcium Chloride × 2 H₂O | 5.28 | g |
| 3 | RO-Water, ad. | 748.99 | mL |
| | | 962 | g |
| Solution B (Sterile Additions) | | | |
| 4 | Potassium Dihydrogen Phosphate | 7.8 | g |
| 5 | Di-Potassium Hydrogen Phosphate | 11.9 | g |
| 6 | Sodium Citrate × 2 H₂O | 66 | g |
| 7 | Magnesium Sulfate × 7 H₂O | 26.4 | g |
| 8 | Trace Element Solution | 13.2 | mL |
| | | 14.65 | g |
| 9 | RO-Water, ad. | 238.88 | mL |
| | | 292.03 | g |
| 10 | NaOH 10% | 14.4 | mL |
| | | 15.97 | g |

After 9 h of exponential feed rate (60.48 to 573.78 g/h), the feed rate was held constant at a rate of 573.78 g/h for the rest of the fermentation (11.5 h). A 60 min temperature ramp (from 33.5° C. to 30.0° C.) was initiated 11.5 h after the start of the exponential feed, and the ramps were completed directly before induction with IPTG was carried out. The culture was induced via a bolus of IPTG, 12.5 h after feed start. The MC was terminated 20.5 h after feed start. At the end of the culturing process, the culture broth was immediately cooled to <12° C., diluted with reverse osmosis (RO) water to a target wet cell weight (WCW) of 15% and bacterial cell mass was separated from the supernatant via centrifugation with a CEPA centrifuge. Biomass was harvested and, together with the supernatant, and transferred to downstream processing.

Product quantification was performed by using the LabChip GXII® system (Perkin Elmer) which provides is an automated high-throughput alternative to traditional SDS-PAGE and protein quantification. Sample preparation was performed with a liquid handling system (Tecan Freedom EVO 150). For product quantification from fermentation samples, analytical cell disruption of samples from fermentations was facilitated via enzymatic cell lysis. 90 μL of fermentation suspension was diluted in a 9:10 ratio (v/v) with cell disruption buffer (Lysonase™ (Merck) in FastBreak™ cell lysis reagent (Promega) with 32 μL Lysonase per 1 mL FastBreak™ reagent). For the total product determination (soluble and insoluble fraction), the samples were mixed before every pipetting step. Finally, the samples were diluted into the specific sample buffer of the system. To minimize the amount of required sample buffer, all dilution steps were carried out in PBS or another formulation buffer.

For the final dilution, 8 μL sample (from the PBS dilutions) or the standard curve samples were diluted in 28 μL non-reducing sample buffer in a 96-well plate (Eppendorf twin.tec PCR plate 95100401). For reducing conditions, 28 μL reducing sample buffer (with 35 mM DTT) was used. The plate was sealed with foil (Eppendorf PCR foil 0030127790), briefly centrifuged (30 s at 25 g) and denatured for 10 min at 70° C. After denaturation, the plate was centrifuged for 5 min at 2200 g to spin down any evaporated liquid. After centrifugation, the foil was removed and diluted via 140 μL DI water. The 96-well plate was sealed with a foil (Eppendorf twin.tec PCR plate 95100401) and the plate was centrifuged for 10 min at 2200 g to sediment any potential aggregates that would cause a malfunction of the LabChip analysis. After centrifugation, the plate was analyzed in the LabChip GXII with the setting “HT Protein Express 100 High Sensitivity”. The LabChip preparation was carried out according to the manufacturing guide. The standard curve was prepared by diluting reference material. As reference material, the [+G] variant was used that had been produced in HEK 293-6E cells as described in Polten (2019), see page 1303, 1^{st} column and Figure S1, and Ebenhoch (2019), see page 8 col. 1.

The quantification was carried out in the range from 1 mg/mL to 0.1 mg/mL via a linear fit. Reducing and non-reducing conditions did not change the integral area for quantification, however a shift in the running time was observed that did not influence the quantification.

### 1.3.3 Purification and Analysis of Inclusion Bodies

Frozen E. coli biomass obtained as described above was resuspended 1:5 (w/v) with IB prep buffer 1 (1 M Urea, 50 mM Tris, 0.1% (v/v) Polysorbat 20, pH 7.5). Following 15 min re-suspension with an ultraturrax, E. coli cells were disrupted by high-pressure homogenization using 3 passes at 650-700 bar. Dense and heavy inclusion bodies (IBs) as well as large cell fragments were separated by high speed tubular centrifugation using a GLE rotor (CEPA). The feed flow rate was 55 mL/min and the centrifugation speed 24,500 g. The tubing had an inner diameter of 3.2 mm. The recovered pellet was washed twice with HQ water. In all cases the pellets were diluted 1:5 (w/v) and re-suspended using an ultraturrax. After the HQ water steps the pellet mostly contains IBs.

### 1.3.4 Protein Solubilisation, Refolding and Purification

Frozen IBs obtained as described above were solubilized at room temperature in solubilization buffer (8 M Urea, 0.14 M GuHCl, 6 mM DTT, 50 mM Tris, pH 8). The mixture was first stirred with an ultraturrax for 10 minutes and then with a propeller mixer for 180 minutes. The target concentration during solubilization was 5 mg/mL with a target volume of 100 mL. Subsequently the solubilization pool was filtered over a CUNO depth filter (filter E16E01A90ZB08A, 0.1-0.6 μm, 3M Deutschland GmbH, Neuss, Germany). Filters were pre equilibrated with water for injection (WFI) and solubilization buffer. Subsequently, the solubilization pool was loaded directly. The filtrate was collected by UV monitoring using an ÄKTA system. The inclusion body solubilisate was diluted with 1:5 refold buffer (4 M Urea, 0.3125 M Tris, 12.5 mM CaCl₂, 3.75 mM cystamine, pH 7). The recovered refold pool was stirred overnight. On the next day it was filtered over a CUNO depth filter (filter E16E01A60ZB05A, 3M Deutschland GmbH, Neuss, Germany).

After filtration with the depth filter, the filtrate was subjected to ultrafiltration/diafiltration (UFDF) to perform a buffer exchange. The UFDF was carried out with a Pellicon 3 membrane (88 cm², Ultracell 3 kDa, screen type C) using a diafiltration buffer (20 mM Tris, pH 9). A concentration factor of 2 and a diafiltration factor of 5 were used.

Following UFDF, the filtrate was subjected to ion exchange chromatography (IEX). A YMC Biopro IEX (Q75) 75 μm column was used with a column diameter 1 cm, a bed height of 9 cm, and a column volume of 7.5 ml. The column was first equilibrated for 5 min with 3 column volumes (CV) equilibration buffer 1 (20 mM Hepes, 1 M NaCl pH 7) and subsequently for 5 min with 5 CV equilibration buffer 2 (20 mM Tris pH 9). After loading the filtrate, the filtrate was washed for 5 min with 5 CV 20 mM Tris pH 9. Protein was eluted for 5 min with 5 CV elution buffer 1 (20 mM Hepes, 1 M NaCl, pH 7) followed by 5 min with 10 CV elution buffer 2 (20 mM Hepes, pH 7) using a gradient (0% to 100% 20 mM Hepes, 1 M NaCl pH 7. Finally, the column was stripped for 5 min with 5 CV with 1 M HCl.

### 1.3.5 Protein Yields and Product Homogeneity

Purification was performed with all 4 variants at least once. In addition, a second purification experiment was performed with [+A] and [+S] variants. The Purification resulted in high yield and high purity for each of the four variants. In particular, the overall process yield after purification and refold for the [+A] variant was found to be 2.4 g/L for the first batch and 5.3 g/L for the second batch.

Analytical high performance size exclusion chromatography was performed in order to test for purity. The purified [+A] variant displayed a high purity of 99.75% main peak, 0.25% low molecular weight impurities and 0.0% aggregate levels. Similarly high purity levels were achieved with the [−V] variant (99.64% main peak, 0.0% low molecular weight impurities, 0.04% aggregates) and the [+S] variant (99.73% main peak, 0.2% low molecular weight impurities, 0.05% aggregates). In contrast, the [+G] variant product was less homogeneous when examined with high performance size exclusion chromatography showing 60.36% main peak purity and 39.64% aggregates.

Process yields from lab scale purification runs for all N-terminal variants are summarized in the following table:

TABLE 4
Summary of lab scale production of MYDGF N-terminal variants. Amount
of MYDGF at different process steps are provided in mg MYDGF.

| | | MYDGF variant | | | |
|---|---|---|---|---|---|
| | | [+A] variant | | | |
| | | h[+A]-MYDGF- | [+G] | [−V] | [+S] |
| | | E. coli | variant | variant | variant |
| End of | optical density | 326 | 332 | 348 | 331 |
| fermentation | (OD 550 nm) | | | | |
| cell density | | | | | |
| End of | (amount wet cells | 330.17 | 310.42 | 337.58 | 311.65 |
| fermentation | g per L | | | | |
| wet cell | fermentation | | | | |
| weight | volume) | | | | |
| End of | (amount | 27.1 | 23.1 | 26.8 | 24.2 |
| fermentation | MYDGF: mg per | | | | |
| product titer | L fermentation | | | | |
| | volume) | | | | |

The fed batch fermentation process applied for all four variants resulted in very high cell densities at end of fermentation (OD at 550 nm of 326-348; wet cell mass of 310,42-337,58 g/L). Very high volumetric titers for recombinant MYDGF variants were achieved (23,1-27,1 g/L fermentation).

### Example 2: Advanced MYDGF Manufacturing Process

Based on Example 1.3.2-1.3.5 the manufacturing process was further developed for the h[+A]-MYDGF-E.coli MYDGF variant. The fermentation process for MYDGF was first developed at 5 L scale using the Research Cell Bank (RCB), then verified by consolidation runs at 20 L scale using GMP working cell bank (WCB) and finally transferred to 200 L scale. A typical 200 L fermentation batch yields 16-18 kg wet IBs.

The downstream process for purification of MYDGF drug substance from intracellular inclusion bodies was developed first at laboratory scale, then verified by consolidation runs at pilot scale using inclusion bodies from a 10 L fermentation aliquot and finally transferred to a cGMP facility where one downstream batch starts with 10 kg wet IBs representing a fermentation aliquot of about 110-125 L.

TABLE 5
Description of the CMC1a drug substance manufacturing
process (upstream process part)

| Process | | Primary function |
|---|---|---|
| step | Description of the process step | of the process step |
| Cell Bank | Manufacturing of Master | |
| | Cell Bank (MCB) | |
| | and Working Cell Bank | |
| | (WCB). MCB is | |
| | derived from Research Cell | |
| | Bank (RCB). | |
| Seed Culture 1 | Seed culture (SC) is performed in | generate sufficient inoculum |
| | shake flasks containing 310 mL | for main culture |
| | sterilized medium. Sterilized seed | |
| | culture medium is inoculated with | |
| | 100 μl of WCB. The seed culture is then | |
| | cultivated and terminated based on the | |
| | OD550 in one OD reference flask. | |
| | The SC is transferred to main culture. | |
| Main Culture | The main culture is conducted as a | expand seed culture and |
| | fed-batch fermentation. | expression of target protein |
| | Product formation is induced by a | |
| | bolus addition of an IPTG-solution. | |
| Biomass | Fermentation broth is cooled down | recovery and concentration |
| Harvest | and biomass is harvested through | of host cells |
| | centrifugation using a tubular bowl | |
| | centrifuge. | |
| Resuspension | The biomass is resuspended and | Resuspension of harvested |
| of Biomass | diluted with a resuspension buffer. | biomass |
| Homogenization | The resuspended biomass is | Disruption of E. coli cells |
| | homogenized in three passes a high- | |
| | pressure homogenizer at 650 bar. The | |
| | lysate is collected in a chilled mobile | |
| | tank. | |
| Harvest of IBs 1 | Harvest of Inclusion Bodies | Harvesting inclusion bodies |
| | (=Centrifugation of Homogenate) is | from homogenate |
| | performed with a tubular bowl | |
| | centrifuge. | |
| IB Wash 1 | IBs 1 are collected after centrifugation | Washing inclusion bodies 1 |
| | of homogenate and diluted 1:5 (w/v) | to remove cell debris and |
| | in purified water. | impurities |
| Harvest of IBs 2 | Harvest of Inclusion Bodies | Harvesting inclusion bodies |
| | (=Centrifugation of Homogenate) is | 2 from inclusion bodies 1 |
| | performed with a tubular bowl | |
| | centrifuge. | |
| IB Wash 2 | IBs 1 are collected after centrifugation | Washing inclusion bodies 2 |
| | of homogenate and diluted 1:5 (w/v) | to remove cell debris and |
| | in purified water. | impurities |
| Harvest of final | Harvest of Inclusion Bodies | Harvest of final |
| Inclusion Bodies | (=Centrifugation of Homogenate) is | inclusion bodies |
| | performed with a tubular bowl | |
| | centrifuge. | |
| Packaging of | The final IB pellet is packed into 500 | Packaging of final Inclusion |
| final IBs | g IB aliquots and stored at −20° C. until | Bodies into disposable |
| | further processing. | containers |

TABLE 6
Description of the CMC1a drug substance manufacturing process (downstream part)

| | | Primary function |
|---|---|---|
| Process step | Description of the process step | of the process step |
| Solubilization | Frozen Inclusion bodies are | Solubilization of frozen |
| | solubilized at 32 g IB/L target | inclusion |
| | concentration using a buffer | bodies |
| | containing | |
| | high chaotrope concentration and | |
| | reducing conditions. | |
| Depth Filtration 1 | The IB solution is filtered through a | Clarification of the IB |
| | depth filter. | solution |
| | | Reduce particles |
| Refolding | The solubilized and filtered IB | Formation of native like |
| | solution is refolded by adding refold | protein + A MYDGF variant |
| | buffer in a ratio of 1:4 and subsequent | |
| | incubation under constant stirring. | |
| UFDF1 | The refold is first concentrated with a | Reduction of the process |
| | factor of 1.5, followed by continuous | volume |
| | diafiltration with at least | Removal chaotropes and |
| | 5 diavolumes. | refolding additives. |
| | | Decrease conductivity |
| Depth Filtration 2 | The UFDF1 pool is filtered through a | Clarification of the IB |
| | depth filter. | solution |
| | | Depletion of host related |
| | | impurities and product |
| | | aggregates |
| AEX | The filtered UFDF1 pool is loaded | Volume reduction |
| chromatography | onto an anion exchange | Depletion of process related |
| | chromatography column. | and product related |
| | Unbound impurities are removed by | impurities |
| | washing with low salt buffer. | |
| | The product is recovered by gradient | |
| | elution. | |
| HIC | The AEX pool is first diluted with | Depletion of process related |
| chromatography | buffer containing high salt | and product related |
| | concentration. The resulting HIC load | impurities |
| | is then loaded onto a hydrophobic | |
| | interaction chromatography column. | |
| | Unbound impurities are removed by | |
| | washing with high salt buffer. | |
| | The product is recovered by gradient | |
| | elution. | |
| UFDF 2 | The HIC pool is first concentrated to a | Concentration of the product |
| | target concentration of ~20 g/L using | Depletion of process related |
| | a membrane with a nominal cut off | impurities of low molecular |
| | of 5 kDa. | weight. |
| | Then the retentate is diafiltered with a | |
| | sodium chloride solution | |
| | (5 diavolumes) followed by | |
| | 6 diavolumes of 20 mM Tris-HCl, pH | |
| | 8.5. | |
| | The retentate is then concentrated to a | |
| | concentration >70 g/L and captured | |
| | from the system. | |
| Formulation | 5-fold formulation buffer is added to | Adjust excipient |
| | the UFDF 2 diafiltrate 2. | concentration and |
| | | reach 50 ± 5 g/L, pH 8.5 ± |
| | | 0.2 |
| DS filtration and | Filtration using a pre-sterilized | Bioburden removal |
| aliquotation | manifold equipped with | Packaging and storage |
| | a 0.2 μm filter; the bulk drug | |
| | substance is filtered into a bag for | |
| | homogenization; | |
| | aliquotation of DS into 6 L bags; | |
| | DS is frozen at <−60° C. to ≥−80° C. | |
| | stored at −40 ± 5° C. | |

Several batches were performed under GMP conditions. The batches resulted in high yields of typically 330-355 g MYDGF from one 125 L fermentation aliquot. This reflects an overall process yield of up to 2.84 g/L fermentation. The MYDGF drug substance produced by this process fulfilled all quality requirements necessary for the use in toxicological and clinical studies.

Monomer content measured by HP size exclusion chromatography was routinely above 99% with high molecular weight impurities (aggregates) below 1% and low molecular weight impurities (fragments) below 0.1%. Endotoxin content was below 0.03 EU per mg of the MYDGF protein.

Host cell DNA content was ≤ 3 pg/mg protein.

The excipients, pH and concentration of the MYDGF protein have to be adapted according to the specifics of the formulations described herein.

### Example 3: Molecular Weight Analysis by LCMS and Advanced Molecular Weight Analysis by LCMS After Chemical Mdification according to Tolonen, A.C. et al. (2011), (“aLCMS”)

Samples of the folded and purified product obtained from Example 2 were subjected to Liquid Chromatography Mass Spectrometry (LCMS) analysis. Liquid Chromatography/Electrospray ionization Mass Spectrometry (LC-ESI-MS) was used to perform intact (non-reduced) molecular weight analysis on MYGDF constructs to (1) verify the sequence via conformity of the observed molecular weight to the predicted values for each sequence, and (2) capture a global profile of the net post-translational modifications (PTMs) on each protein. An Agilent 1290 UPLC with a 1.0 mm by 30 mm C3 POROS reversed phase column was used to desalt and introduce samples (0.5μg/injection) into the mass spectrometer. A three minute binary gradient consisting of mobile phase A (98.9% water, 1% acetonitrile, 0.1% formic acid, and 2 mM ammonium acetate) and mobile phase B (70% isopropanol, 20% acetonitrile, 9.9% water, and 0.1% formic acid) that increased from 5% to 80% of mobile phase B at 150 μl/min was used to trap, desalt, and elute the protein from the column. Mass spectral data of the eluted material were acquired using an Agilent 6224 Time-of-Flight (TOF) MS, which was then processed (deconvoluted) using the maximum entropy algorithm within the Mass Hunter analysis software (Agilent). Data obtained by this method is referred to herein as “intact MW LCMS data” or data “measured by liquid chromatography mass spectrometry (LCMS)”.

For peptide-level sequence confirmation and site-specific post-transcriptional modification (PTM) analysis, aliquots of each sample were digested using trypsin and chymotrypsin separately to achieve complete sequence coverage. 100 μg of each sample was desalted and concentrated via acetone precipitation and centrifugal pelleting of the precipitated material. Each protein pellet was re-solubilized, denatured, and reduced in 10μl of denaturation/reduction buffer (5% w/v sodium deoxycholate (SDC), 10 mM dithiothreitol (DTT), 20 mM ammonium bicarbonate) and incubated at 70° C. for 2 minutes, followed by a ten-fold dilution with 20 mM ammonium bicarbonate and 2 mM methionine. The reduced/denatured molecules were then split into two vials (50 μg each), whereupon trypsin and chymotrypsin were added separately at a 1:10 enzyme-to-substrate ratio to each tube, and the samples incubated for 10 minutes at 37° C. The reaction was quenched with addition of 10% v/v trifluoroacetic acid, resulting in 1% v/v final concentration of that reagent. The short (10 min) digestion step obviated the need for an alkylation step which is commonly used in peptide mapping. Precipitated sodium deoxycholate was removed by centrifugation at 16,000×g, and the peptide-containing supernatant was recovered and transferred to autosampler vials which were immediately stored at −80° C. until analysis. Data obtained by this method is referred to herein as “peptide mapping LCMS data”

aLCMS: Additionally, as the first four N-terminal residues of the various MYGDF constructs have been shown to undergo fragmentation during electrospray (both at the intact and peptide levels), reductive dimethylation (also known as stable isotope dimethyl labeling (SIDL)) was performed according to Tolonen et al. (2019), on aliquots of the peptide digests to delineate between sample-derived versus electrospray-derived N-terminal truncations. Data obtained by this method is referred to herein as “LCMS after reductive dimethylation (stable isotope dimethyl labelling, SIDL)”. Briefly, 50 μg of peptides from each digest were immobilized into separate Waters Oasis SPE cartridges using a vacuum manifold. The SPE media and peptides were conditioned to pH 5.5 with citrate buffer (90 mM citric acid, 230 mM divalent sodium phosphate), and 10 ml of 0.8% v/v formaldehyde (in citrate buffer) and 120 mM sodium cyanoborohydride was then passed over the bound peptides for 10 minutes. The reactants were then removed by washing with 10 column volumes of 0.1% formic acid in water, and eluted with 10 volumes of 50% acetonitrile, 0.1% formic acid. The labeled peptides were collected into a low-retention microcentrifuge tube were then taken to dryness in a vacuum centrifuge. The dried peptides were re-constituted in 50 μl of 0.1% TFA and transferred to an autosampler vial for LC-MS/MS analysis. LC-MS/MS (tandem mass spectrometry) analysis was performed using a Vanquish UHPLC system interfaced with a Lumos Fusion Orbitrap (ThermoFisher) that was operated under the control of Xcalibur 4.1.31.9 software (ThermoFisher). 0.5 μg of each peptide digest was loaded onto a 2.1 mm×150 mm C18 CSH Acquity UPLC reversed phase column (1.7 μm particle, Waters Corp.), and separated using a binary gradient as follows: (mobile phase A=0.1% difluoroacetic acid (DFA) in water) 0.5% to 40% of mobile phase B (99.9% acetonitrile, 0.1% DFA) at a flow rate of 200 μl/min and column temperature of 50° C. A top-4 data-dependent acquisition (DDA) MS workflow was used to analyze the LC eluate. Full scan MS spectra were acquired at 120,000 resolution (FWHM) at 200 m/z, and HCD (high energy collisional dissociation) and EThcD (electron transfer dissociation with supplemental HCD energy) MS/MS spectra were acquired in a charge-state dependent manner at 15,000 resolution in the Orbitrap analyzer. The resultant .RAW files from each LC-MS/MS analysis were further processed using Protein Metrics Inc., (PMI) Byonic and Byos software to identify and quantify PTMs. Manual analysis of various spectra was performed using the QualBroswer feature of Xcalibur software.

TABLE 7
MYDGF variants examined by aLCMS or LCMS:

| | MYDGF ( . . . ) | Host | Expression |
|---|---|---|---|
| No. | type | system | type |
| 1 | +S . . . variant (h[+S]-MYDGF-Ecoli) | E. coli | Insoluble |
| 2 | −V . . . variant (h[−V]-MYDGF-Ecoli) | E. coli | Insoluble |
| 3 | +A . . . variant (h[+A]-MYDGF-Ecoli) | E. coli | Insoluble |
| 4 | +G . . . variant (h[+G]-MYDGF-Ecoli) | E. coli | Insoluble |
| 5 | wt | CHO | Soluble |

TABLE 8
Intact LCMS MW data for +S MYDGF variant

| | PTM | | Δ MW | Predicted | Observed | Relative |
|---|---|---|---|---|---|---|
| Sample | category¹ | Description | (Da) predicted | MW (Da) | MW (Da) | percent |
| +S | Full | unmodified | 0 | 15,920 | 15,920 | 80.9 |
| | length | | | | | |
| | N-term | +M | 131 | 16,051 | 16,051 | 3.6 |
| | addition | | | | | |
| | N- | −SVSE | −402 | 15,518 | 15,518 | 1.5 |
| | term trunc | −SVS | −258 | 15,646 | 15,647 | 0.2 |
| | ations | −SV | −186 | 15,733 | 15,734 | 1.6 |
| | | −S | −87 | 15,832 | 15,833 | 0.6 |
| | Additional | Dehydration | −18 | 15,902 | 15,902 | 1.4 |
| | PTMs | Na+ | 22 | 15,942 | 15,941 | 3.7 |
| | | Carbamoylation | 43 | 15,963 | 15,962 | 4.5 |
| | | gluconoylation | 178 | 16,098 | 16,098 | 2.0 |

(¹PTMs = Post Translational Modifications). The Na⁺ adduct in intact analysis is a common artefact, not a molecular attribute. The term “N-term” in the peptide map refers to the amino group of the N-terminus. The sequence coverage is 100%.
N.D. = PTM < lower limit of detection

TABLE 9
Peptide Mapping LCMS data for +S MYDGF variant

| | | | | Non-binding |
|---|---|---|---|---|
| | | Binding region | | region |
| Potential modification | | Percent | | Percent |
| | Residue | modification | Residue | modification |
| Sequence variants | n.a. | n.a. | N-term | 84.6* |
| | | | unmodified | |
| | | | N-term + M | 3.6* |
| | | | N-term − SVSE | 1.5* |
| | | | N-term − SVS | 0.2* |
| | | | N-term − SV | 1.6* |
| | | | N-term − S | 0.6* |
| Oxidation (16) | Y70, Y72 | N.D. | M34 | 0.5 |
| | | | M49 | 0 |
| | | | M91 | 1.3 |
| | | | W47 | N.D. |
| | | | W65 | N.D. |
| Isomerization (0) | n.a. | n.a. | D11 | N.D. |
| | | | D29 | |
| | | | D56 | |
| | | | D103 | |
| Gluconoylation (178) | K70 | N.D. | N-term | 1.4 |
| | K97 | | K28 | N.D. |
| | K107 | | K77 | N.D. |
| | K126 | | K113 | N.D. |
| | K131 | | K135 | N.D. |
| Carbamoylation (43) | K70 | N.D. | N-term | 3.1 |
| | K97 | | K28 | N.D. |
| | K107 | | K77 | N.D. |
| | K126 | | K113 | N.D. |
| | K131 | | K135 | N.D. |
| Acetylation (42) | K70 | N.D. | N-term | 2.4 |
| | K97 | | K28 | N.D. |
| | K107 | | K77 | N.D. |
| | K126 | | K113 | N.D. |
| | K131 | | K135 | N.D. |
| Dehydration | n.a. | n.a. | D29 | 0 |
| (succinimide) (−18) | | | D56 | 4.2 |
| Methylation/ | K70 | N.D. | N-term | N.D. |
| glycation/ | K97 | | K28 | N.D. |
| glycosylation | K107 | | K77 | N.D. |
| | K126 | | K113 | N.D. |
| | K131 | | K135 | N.D. |

*determined form intact Mw analysis. The Na⁺ adduct added to original value

TABLE 10
Intact LCMS MW data for −V MYDGF variant

| | PTM | | Δ MW | Predicted | Observed | Relative |
|---|---|---|---|---|---|---|
| Sample | category | Description | (Da) predicted | MW (Da) | MW (Da) | percent |
| −V | Full length | unmodified | 0 | 15,733 | 15,734 | 74.0 |
| | N-term | −SE | −216 | 15,517 | 15,518 | 3.7 |
| | truncations | | | | | |
| | Additional | Dehydration | −18 | 15,715 | 15,716 | 1.9 |
| | PTMs | Na+ | 22 | 15,755 | 15,754 | 4.4 |
| | | Carbamoylation | 43 | 15,776 | 15,776 | 12.1 |
| | | gluconoylation | 178 | 15,911 | 15,912 | 3.9 |

TABLE 11
Peptide Mapping LCMS data for −V MYDGF variant

| | Binding region | | Non-binding region | |
|---|---|---|---|---|
| Potential | | Percent | | Percent |
| modification | Residue | modification | Residue | modification |
| Sequence variants | n.a. | n.a. | N-term | 78.4* |
| | | | unmodified | |
| | | | N-term − SE | 3.7* |
| Oxidation (16) | Y70, Y72 | N.D. | M32 | 2.5 |
| | | | M47 | 3.9 |
| | | | M89 | N.D. |
| | | | W45 | 1.2 |
| | | | W63 | N.D. |
| Isomerization (0) | n.a. | n.a. | D9 | N.D. |
| | | | D27 | |
| | | | D54 | |
| | | | D101 | |
| Gluconoylation (178) | K68 | N.D. | Prot. N-term | 6.7 |
| | K93 | | K28 | N.D. |
| | K105 | | K77 | N.D. |
| | K124 | | K113 | N.D. |
| | K129 | | K135 | N.D. |
| Carbamoylation (43) | K68 | N.D. | Prot. N-term | 9.5 |
| | K93 | | K28 | N.D. |
| | K105 | | K77 | N.D. |
| | K124 | | K113 | N.D. |
| | K129 | | K135 | N.D. |
| Dehydration | n.a. | n.a. | D27 | 3.1 |
| (succinimide) (−18) | | | D54 | 0.1 |
| Acetylation/ | K68 | N.D. | Prot. N-term | N.D. |
| methylation/ | K93 | | K28 | |
| glycation/ | K105 | | K77 | |
| glycosylation | K124 | | K113 | |
| | K129 | | K135 | |

*determined from intact MW analysis; Na+ adduct added to original value

TABLE 12
Intact MW LCMS data for +A MYDGF variant

| | | | Δ MW | Predicted M | Observed M | Relative |
|---|---|---|---|---|---|---|
| Sample | PTM category | Description | (Da) predicted | W (Da) | W (Da) | percent |
| +A | Full length | unmodified | 0 | 15,904 | 15,904 | 83.1 |
| | N-term | −AVSE | −387 | 15,517 | 15,518 | 1.5 |
| | truncations | −AVS | −258 | 15,646 | 15,647 | 0.1 |
| | | −AV | −171 | 15,733 | 15,733 | 0.5 |
| | | −A | −71 | 15,832 | 15,833 | 0.4 |
| | Additional | Dehydration | −18 | 15,886 | 15,886 | 1.7 |
| | PTMs | Na+ | 22 | 15,926 | 15,924 | 5.6 |
| | | Carbamoylation | 43 | 15,947 | 15,946 | 4.6 |
| | | gluconoylation | 178 | 16,082 | 16,082 | 2.5 |

TABLE 13
Peptide Mapping LCMS data for +A MYDGF variant

| | Binding region | | Non-binding region | |
|---|---|---|---|---|
| Potential | | Percent | | Percent |
| modification | Residue | modification | Residue** | modification |
| Sequence variants | n.a. | n.a. | N-term | 88.7* |
| | | | unmodified | |
| | | | N-term − AVSE | 1.5* |
| | | | N-term − AVS | 0.1* |
| | | | N-term − AV | 0.5* |
| | | | N-term − A | 0.4* |
| Oxidation(16) | Y72, | N.D. | M33 | 0.3 |
| | Y74 | | M48 | 0.3 |
| | | | M91 | 1.4 |
| | | | W47 | 1.4 |
| | | | W65 | N.D. |
| Isomerization (0) | n.a. | n.a. | D11 | N.D. |
| | | | D29 | |
| | | | D56 | |
| | | | D103 | |
| Gluconoylation (178) | K70 | N.D. | Prot. N-term | 1.9 |
| | K95 | | K30 | N.D. |
| | K107 | | K79 | N.D. |
| | K126 | | K115 | N.D. |
| | K131 | | K137 | N.D. |
| Carbamoylation (43) | K70 | N.D. | Prot. N-term | 2.1 |
| | K95 | | K30 | N.D. |
| | K107 | | K79 | N.D. |
| | K126 | | K115 | N.D. |
| | K131 | | K137 | N.D. |
| Dehydration | n.a. | n.a. | D29 | 2.8 |
| (succinimide) (−18) | | | D56 | 3.3 |
| Acetylation/ | K70 | N.D. | Prot. N-term | N.D. |
| methylation/ | K95 | | K30 | |
| glycation/ | K107 | | K79 | |
| glycosylation | K126 | | K115 | |
| | K131 | | K137 | |

*determined form intact mass analysis Na+ adduct added to original value
**N-terminal methionine was not observed

TABLE 14
Intact MW LCMS data for +G MYDGF variant

| | PTM | | Δ MW | Predicted MW | Observed MW | Relative |
|---|---|---|---|---|---|---|
| Sample | category | Description | (Da) predicted | (Da) | (Da) | percent |
| +G | Full length | unmodified | 0 | 15,889 | 15,890 | 37.1 |
| | Additional | Dehydration | −18 | 15,871 | 15,872 | 0.8 |
| | PTMs | Na+ | 22 | 15,911 | 15,910 | 2.3 |
| | | Carbamoylation | 43 | 15,932 | 15,932 | 4.2 |
| | N-term | +Met | 131 | 16,021 | 16,021 | 44.1 |
| | addition | Dehydration | −18 | 16,003 | 16,003 | 1.0 |
| | | Na+ | 22 | 16,043 | 16,040 | 3.4 |
| | | Carbamoylation | 43 | 16,064 | 16,067 | 7.1 |

TABLE 15
aLCMS Data (combined Intact MW LCMS and dimethyl-capped peptide level LCMS after trypsination as described*:

| R&D Systems, rMYDGF | | | | | | | | |
|---|---|---|---|---|---|---|---|---|
| (human, wt, untagged) | | | (+A) MYDGF | | | (+S) MYDGF | | |
| Cat. No. 10231 | | | variant | | | variant | | |
| | | Percent | | | Percent | | | Percent |
| | | N-term | | | N-term | | | N-term |
| | | cleavage | | | cleavage | | | cleavage |
| | | verified by | | | verified by | | | verified by |
| | Whole | peptide | | Whole | peptide | | Whole | peptide |
| | protein | mapping and | | protein | mapping and | | protein | mapping and |
| N-term | analysis | aLCMS | | analysis | aLCMS | | analysis | aLCMS |
| modification | Percent | methodology | | Percent | methodology | | Percent | methodology |
| unmodified | 100.0 | 99.9 | unmodified | 93.4 | 93.2 | unmodified | 93.8 | 93.3 |
| −V | | 0.0 | −A | | 0.2 | −S | | 0.5 |
| −VS | | 0.1 | −AV | | 0.0 | −SV | | 0.0 |
| −VSE | | 0.0 | −AVS | | 0.0 | −SVS | | 0.0 |
| −VSEP | | 0.0 | −AVSE | | 0.0 | −SVSE | | 0.0 |
| Carbamoylation | 0 | | Carbamoylation | 4.3 | | Carbamoylation | 4.2 | |
| Gluconoylation | 0 | | Gluconoylation | 2.3 | | Gluconoylation | 1.9 | |

*N-terminal methionine was not observed in the +A variant; N-terminal methionine was however observed in the +S variant at 2.5%.

### Example 4: Study of Conformational Stability

Thermal stability in the pH range of 5.75 to 8.00 was evaluated by monitoring the thermal unfolding temperature Tm. The conformational stability at different pH values was assessed by differential light scattering. The samples were prepared with different buffers with a concentration of 20 mM and 50 mg/mL protein (h [+A]-MYDGF-E. coli). Thermal stability data indicated that the conformational stability was slightly increasing with increasing pH. Table 16 shows the buffers that were evaluated and the corresponding Tm.

TABLE 16
pH/Buffer Screen: Tₘ values determined

| Buffer | pH | Tₘ (° C.) |
|---|---|---|
| 20 mM acetate | 5.75 | 61.6 |
| 20 mM histidine | 5.75 | 61.0 |
| 20 mM tromethamine | 7.00 | 62.0 |
| 20 mM tromethamine | 8.00 | 63.8 |
| 20 mM phosphate | 8.00 | 63.0 |

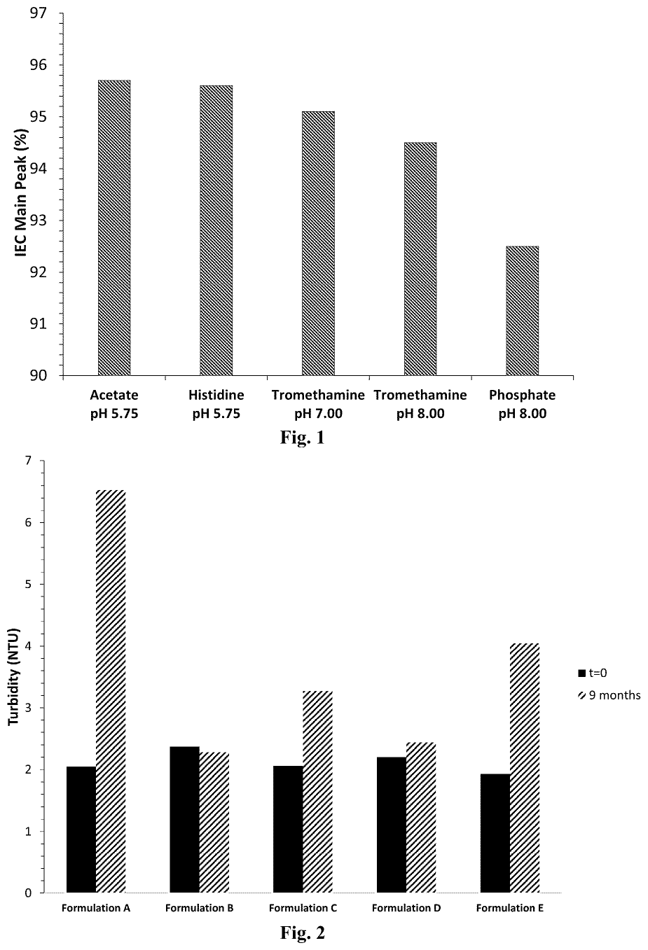

Data of ion exchange chromatography (IEC) after storage of two weeks at 40° C. is depicted in FIG. 1. Values of main peak prior to storage were for all samples 96.7 to 96.9%. Towards the expectation based on the conformational stability, lower pH resulted in less protein degradation detected by IEC after storage of two weeks at 40° C.

Example 5: pH study

The stability of the h[+A]-MYDGF-E. coli protein of the present invention was evaluated as a function of pH/buffer. Samples were prepared at 50 mg/mL protein at low pH around 6 and at high pH, pH 8.0. Details of the composition of the formulations are summarized in Table 17. The solution was filled into 6 mL glass vial (2.6 mL per vial).

TABLE 17
Formulations evaluated in pH study

| Formulation | Buffer | Excipient 1 | Excipient 2 | Surfactant | pH |
|---|---|---|---|---|---|
| Formulation A | 20 mM | 220 mM | 10 mM | 0.04% (w/v) | 8.0 |
| | tromethamine | sucrose | methionine | polysorbate 80 | |
| Formulation B | 20 mM | 220 mM | 10 mM | 0.03% (w/v) | 6.2 |
| | histidine | sucrose | methionine | polysorbate 20 | |
| Formulation C | 20 mM | 220 mM | 10 mM | 0.03% (w/v) | 6.0 |
| | histidine | sucrose | methionine | polysorbate 20 | |
| Formulation D | 20 mM | 220 mM | 10 mM | 0.04% (w/v) | 6.0 |
| | histidine | sucrose | methionine | polysorbate 80 | |
| Formulation E | 20 mM | 220 mM | 10 mM | 0.03% (w/v) | 5.7 |
| | histidine | sucrose | methionine | polysorbate 20 | |

Surprisingly the study results showed that formulations with the h[+A]-MYDGF-E. coli protein at pH 5.7, 6.0 and 6.2 (20 mM histidine) exhibited the most promising properties. After 9 months storage at 25° C. the turbidity of all histidine formulations (formulation: B, C, D, E) were not more than 4 NTU. For the tromethamine formulation (formulation A) the turbidity increased significantly to 6.5 NTU after storage (FIG. 2).

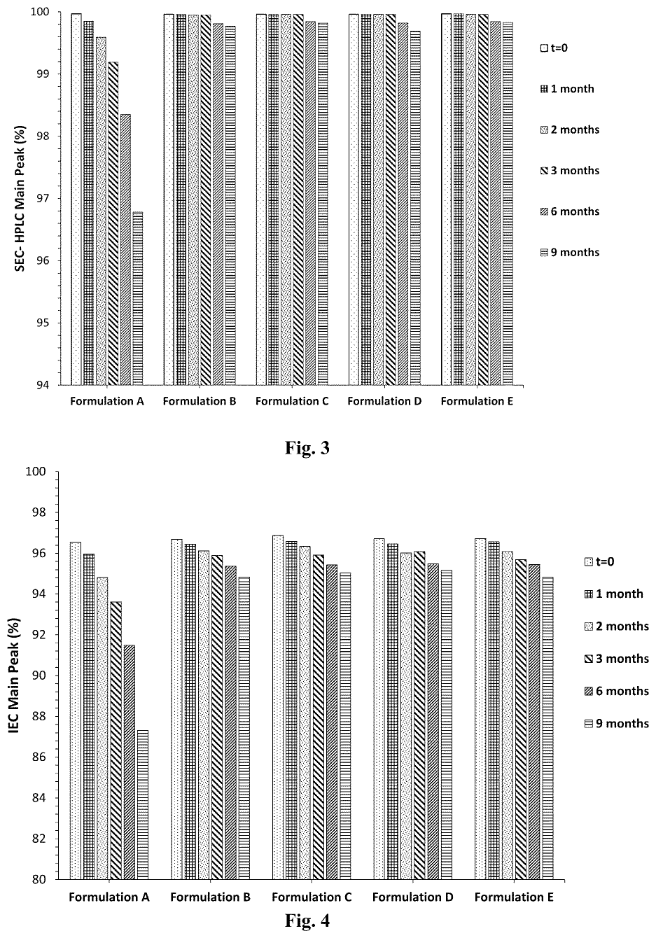

The results of the size exclusion chromatography (SEC-HPLC) are demonstrated by the main peak which represents the monomer content of h[+A]-MYDGF-E. coli (FIG. 3). All histidine formulations showed only minimal reduction of the main peak in the samples stored at 25° C. for up to 9 months. While only minor changes were observed in the histidine containing formulations, the main peak of the tromethamine formulation decreased continuously for more than 3% (formulation A) over 9 months storage at 25° C.

Ion exchange chromatography (IEC-HPLC) results were in line with the SEC-HPLC and turbidity data as only marginal changes were detected up to 9 months storage at 25° C. for all histidine formulations. The main peak represents the purity of the h[+A]-MYDGF-E. coli protein. The main peak dropped for the tromethamine formulation (formulation A) and was significant higher compared to the histidine formulations (decrease of 9.2% vs. 1.9%), results are shown in FIG. 4.

### Example 6: Surfactant Study

This working example was conducted to optimize the concentration of polysorbate in four different formulations containing 20 mM histidine at pH 6 and 50 mg/mL h[+A]-MYDGF-E. coli protein (Table 18). The formulations were filled into 6 mL glass vials (3 mL per vial).

TABLE 18
Formulations evaluated in surfactant optimization study

| Component | Formulation 1 | Formulation 2 | Formulation 3 | Formulation 4 |
|---|---|---|---|---|
| Protein | 50 mg/mL | 50 mg/mL | 50 mg/mL | 50 mg/mL |
| MYDGF | | | | |
| Buffer | 20 mM | 20 mM | 20 mM | 20 mM |
| | histidine | histidine | histidine | histidine |
| Surfactant | 0% (w/v) | 0.02% (w/v) | 0.03% (w/v) | 0.04% (w/v) |
| | polysorbate | polysorbate 20 | polysorbate 20 | polysorbate 80 |

The following experiments were conducted for the surfactant study: freeze-thaw stability, agitation stability. Results of turbidity were assessed.

### Freeze-Thaw Stability Study

The formulations listed in Table 19 were frozen in the glass vials and exposed to five freeze-thaw cycles (−65° C. to 25° C.). Results are summarized in Table 19.

TABLE 19
Turbidity results freeze-thaw study

| | Turbidity | Turbidity after |
|---|---|---|
| | before freeze- | five freeze- |
| | thaw cycles | thaw cycles |
| Surfactant content | NTU | NTU |

| | | | |
|---|---|---|---|
| 0% | (w/v) polysorbate | 3 | 12 |
| 0.02% | (w/v) polysorbate 20 | 2 | 3 |
| 0.03% | (w/v) polysorbate 20 | 2 | 3 |
| 0.04% | (w/v) polysorbate 80 | 2 | 3 |

The formulation without polysorbate (0%) showed an increase in turbidity, all formulations containing polysorbate did not show a relevant change in turbidity. The addition of polysorbate with 0.02% to 0.04% stabilizes the protein during freezing and thawing.

### Agitation Stability

The agitation study was performed with the formulations 1 to 4 with the filled vials on a shaker at 25° C. After even five days of shaking surprisingly in all formulations containing polysorbate (0.02% to 0.04%) no change in turbidity was detected. All turbidity values of formulations with polysorbate remained at 2 NTU (Nephelometric Turbidity Units). In the formulation without polysorbate the turbidity increases from initially 3 NTU to a not measurable value of more than 400 NTU. The addition of polysorbate stabilizes the MYDGF protein during freezing and thawing and protects it during shaking stress.

Additionally, a further study was executed with the formulation of 50 mg/mL protein MYDGF, 20 mM histidine, 10 mM methionine, 220 mM sucrose and varying amounts of polysorbate 80(0%, 0.02%, 0.04% and 0.06% (w/v)) at pH 5.7. The formulations were filled into 20 mL glass vials with a fill volume of 6.2 mL and were shaken at 25° C. for up to seven days. Even after seven days of shaking all vials containing polysorbate did not show any increase in turbidity. All turbidity values of solutions with polysorbate were initially and after seven days equal to or below 3 NTU. The turbidity values of the formulation without polysorbate increased from initially 3 NTU to not measurable values of higher than 400 NTU, already after one day of shaking.

The addition of polysorbate between 0.02% and 0.06% (w/v) protects the MYDGF protein in the formulation upon molecule degradation due to shaking stress.

### Example 7: Long-Term Stability Study

Three formulations (Table 20) were chosen to be tested in a long-term stability study. The formulations with a h[+A]-MYDGF-E. coli protein concentration of 50 mg/mL were stored at 5°° C. for 24 months in type I 6 mL glass vials.

TABLE 20
Formulation compositions for long-term stability study

| Formulation | Buffer | Excipient 1 | Excipient 2 | Surfactant | pH |
|---|---|---|---|---|---|
| Formulation C | 20 mM | 220 mM | 10 mM | 0.03% (w/v) | 6.0 |
| | histidine | sucrose | methionine | polysorbate 20 | |
| Formulation D | 20 mM | 220 mM | 10 mM | 0.04% (w/v) | 6.0 |
| | histidine | sucrose | methionine | polysorbate 80 | |
| Formulation E | 20 mM | 220 mM | 10 mM | 0.03% (w/v) | 5.7 |
| | histidine | sucrose | methionine | polysorbate 20 | |

The stability of each formulation was monitored for 0, 1, 2, 3, 6, 9, 12, 18 and 24 months. The samples were analysed by the following assays: turbidity, subvisible particles, size exclusion chromatography (SE-HPLC), ion exchange chromatography (IEC) and binding.

### Turbidity

The samples were analysed by measurement of turbidity. Surprisingly all three formulations exhibited a very low turbidity from the initial sampling point over the course of the study up to 24 months. All results over the various sampling points were within an extremely tight range from 1.8 to 2.7 NTU.

### Subvisible Particles

The monitoring of subvisible particles in the formulations was performed by light obscuration. Surprisingly, for samples stored till 24 months at 5° C., very low numbers of subvisible particles were observed. Only a maximum of 20 particles per milliliter with diameters ≥10 μm and ≥25 μm were detected (data not shown).

### Size Exclusion Chromatography

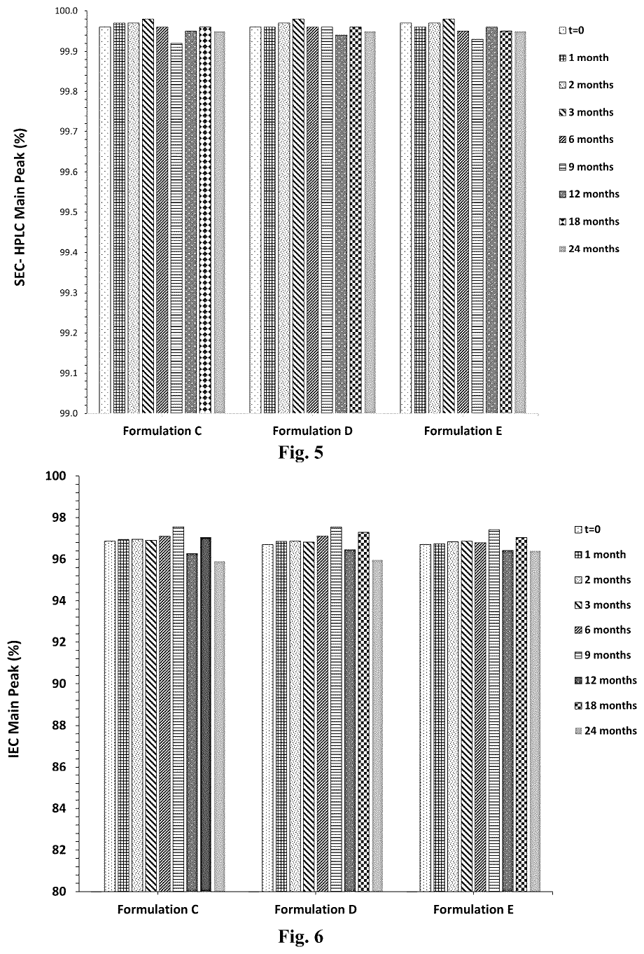

The stability of the formulations was assessed by SEC-HPLC monomer content with evaluation of the main peak. Surprisingly all three formulations showed only negligible changes of the main peak for up to 24 months at 5° C. (FIG. 5).

### Ion Exchange Chromatography

The charge profile was examined by acidic, basic and main peak group measurement by ion exchange chromatography. There was only very little variation of the charge profile of all three formulations up to 24 months. Data of main peak is shown in FIG. 6.

### Binding

The binding of h[+A]-MYDGF-E. coli protein to an anti-MYDGF antibody was assessed using binding assay. Remarkably, no binding loss of all three formulations was observed during storage at 5°° C. for up to 9 months (data not shown).

### Summary of Long-Term Stability Studies

Evaluation of the data from the 24 months stability study showed that formulation C, D and E performed equally well when assessed by various assays. Remarkably, all three formulations showed nearly no change in main peak by size exclusion and ion exchange chromatography over time.

### Example 8: Lyophilisation Study

The formulation E (Table 21) with the h[+A]-MYDGF-E. coli protein concentration of 50 mg/mL is held at 5° C. for 18 months in type I 6 mL glass vials. Stability of this liquid formulation is described in detail in Example 7.

TABLE 21
Composition of formulation E

| Formulation | Buffer | Excipient 1 | Excipient 2 | Surfactant | pH |
|---|---|---|---|---|---|
| Formulation E | 20 mM | 220 mM | 10 mM | 0.03% | 5.7 |
| | histidine | sucrose | methionine | polysorbate 20 | |

Formulation E is developed with the exclusive property that this solution can be lyophilized without any adaptation of the excipient amounts or addition of excipients. The formulation is freeze dried with a standard freeze drying process in glass vials. Although the drug is provided lyophilized, a fast administration is guaranteed by fast reconstitution with water for injection. The reconstitution time of this formulation is extremely rapid with less than 60 seconds. The option to freeze dry the solution offers the opportunity for more extremer shelf-life conditions compared to standard liquid formulations. For example, shelf life can be expanded and long-term storage at ambient temperature is offered for the freeze-dried drug.

The formulation H (Table 22) with the h[+A]-MYDGF-E. coli protein concentration of 50 mg/mL was prepared and filled into 20 mL glass vials with a fill volume of 11 mL. Freeze drying was performed with a standard drying process

TABLE 22
Composition of formulation H

| Formulation | Buffer | Excipient 1 | Excipient 2 | Surfactant | pH |
|---|---|---|---|---|---|
| Formulation H | 20 mM | 220 mM | 10 mM | 0.04% | 5.7 |
| | histidine | sucrose | methionine | polysorbate 20 | |

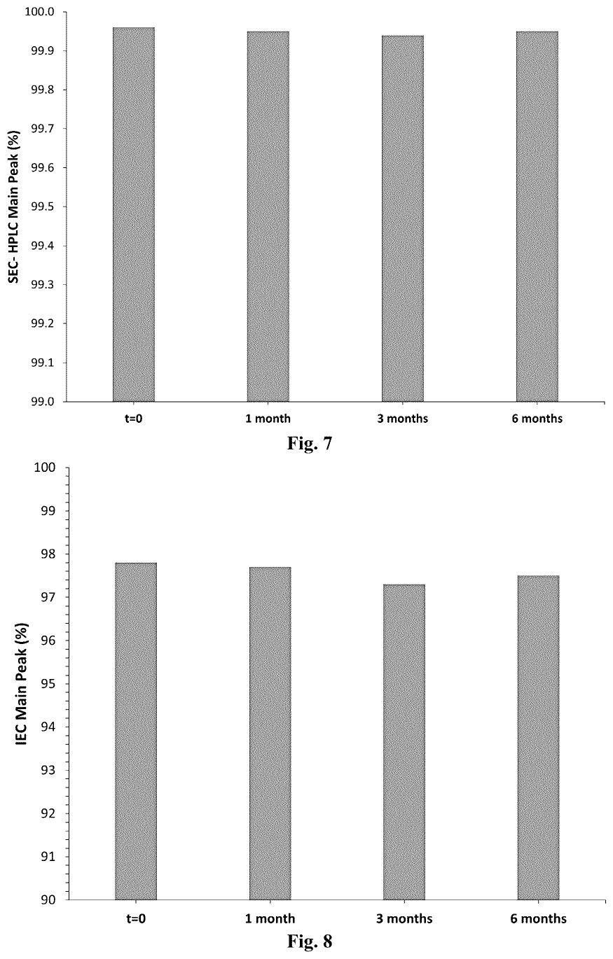

The freeze-dried Formulation H was stored at 25° C. for up to 6 months. Stability was assessed by analysing the monomer content (main peak) by SEC-HPLC and ion exchange chromatography. The results are shown in FIGS. 7 and 8, respectively. It can be seen that no signification degradation of the MYDGF protein could be detected for up to 6 months. Therefore, long-term storage at room temperature is possible.

### Example 9: Lyophilization Feasibility Study

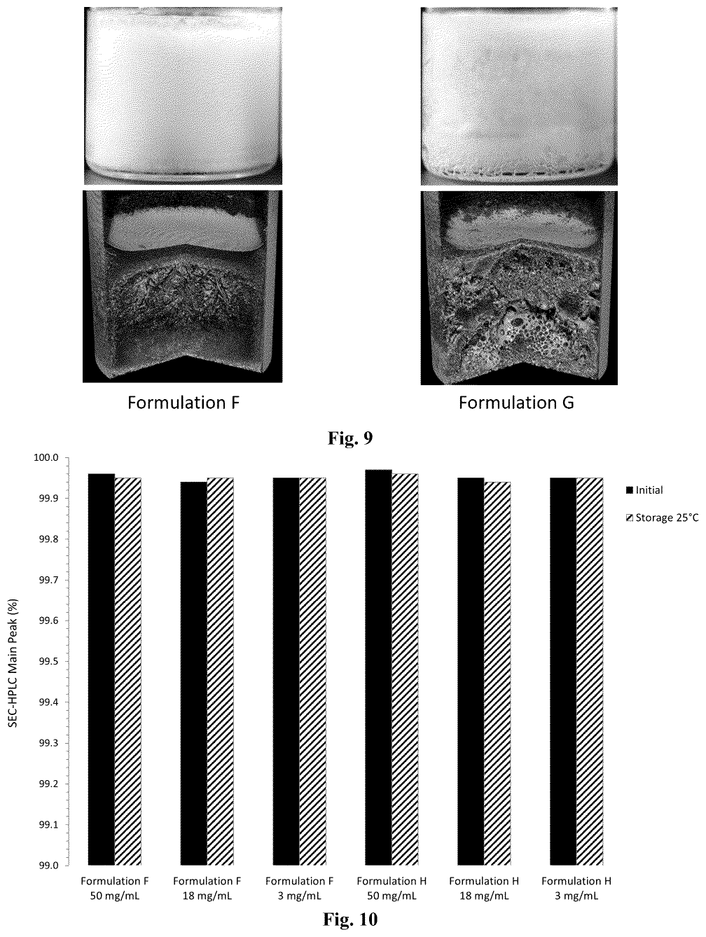

Two different formulations were freeze dried, formulation F and G. Formulation F contains MYDGF protein and the excipients 20 mM histidine, 220 mM sucrose, 10 mM methionine, 0.04% polysorbate 80 at pH 5.7. Formulation G differs from Formulation F by the amount of sucrose which was 440 mM (no change in the amount of methionine, polysorbate 80 and pH). The two solutions, filled into 20 mL glass vials with a fill volume of 12 mL, were lyophilized with the same freeze-drying process. After lyophilization both formulations were visually inspected and underwent microcomputed tomography (μCT) scan in order to evaluate the structure of the lyophilized drug. Pictures and 3-dimentional structure by μCT are depicted in FIG. 9. Significant differences in pore size and even collapsed structure were identified in Formulation G. Collapse in Formulation G is indicated in FIG. 9 by foam-like structure at the bottom of the vial. In the μCT scan, collapse in Formulation G is detected by larger holes. Surprisingly, Formulation F showed an intact structure and more homogeneous pore distribution. This demonstrates that liquid Formulation F can easily be freeze dried.

### Example 10: Dilution Media Compatibility Study

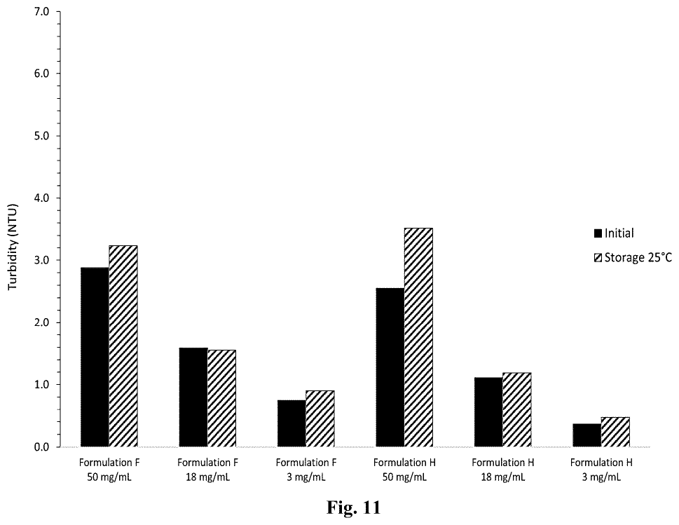

The stability of drug product solution including MYDGF protein without further dilution and diluted with sodium chloride was investigated. The dilution of the drug with 0.9% NaCl solution is a standard procedure for intravenous administration of parenterals. To examine the stability of the drug solution, two formulations were diluted to 18 mg/mL and 3 mg/mL MYDGF protein in 0.9% NaCl solution. The two formulations used were as follows: Formulation F containing 50 mg/mL MYDGF protein, 20 mM histidine, 220 mM sucrose, 10 mM methionine, 0.04% polysorbate 80 at pH 5.7, and Formulation H containing 50 mg/mL MYDGF protein, 20 mM histidine, 220 mM sucrose, 10 mM methionine, 0.04% polysorbate 20 at pH 5.7. Diluted and non-diluted formulations showed no relevant changes in turbidity and SEC-HPLC data after storage for at least 24 hours at 25° C. The data is shown in FIGS. 10 and 11.

### LITERATURE

1. Botnov, V. et al. (2018), J Biol Chem, 293 (34), 13166-13175.

2. Ebenhoch, R. et al. (2019), Nat Commun 10, 5379.

3. Desta L. et al. (2015), JACC Heart Fail, 3 (3), 234-242.

4. Desta L, et al. (2017), Int J Cardiol, 248, 221-226.

5. Korf-Klingebiel, M. et al. (2015), Nat Med 21 (2): 140-9.

6. Korf-Klingebiel, M. et al. (2021), Circulation.144 (15): 1227-40.

7 Polten, F. et al. (2019), Anal Chem, 91, 1302-1308.

8. Tolonen, A.C. et al. (2011), Mol Systems Biol 7 (1).

9. Zhao, L. et al. (2020), J Cell Mol Med, 24 (2): 1189-1199.

10. Frottin, F. et al. (2006), The Proteomics of N-terminal Methionine Cleavage, Molecular & Cellular Proteomics, Volume 5, Issue 12, 2336-2349.

11. Bodenhausen G. & Ruben D. J. (1980), Chem. Phys. Lett. 69, 185.

12. Piotto, M. et al. (1992), J Biomol NMR, 2, 661-666.

13. Sklenar, V. et al. (1993), J Magn Reson, Series A 102, 241-245.

14. Mori, S. et al. (1995), J Magn Reson B 108, 94-98.

15. Wishart, D. S. et al. (1995), J Biomol NMR, 5, 67-81.

16. Dyson, H. J. & Wright, P. E. (1998), Nat Struct Biol, 5, 499-503.

17. Shen Y. & Bax A. (2015), Methods Mol Biol, 1260:17-32.

18. CN 111544572 A

19. EP 2 918 676 B1

20. U.S. Pat. No. 5,744,328 B

21. U.S. Pat. No. 7,851,433 B

22. US 2015/0291683 A1

23. WO 2011/154349 A2

24. Peternel, S. & Komel, R. (2010), Microbial Cell Factories, 9:66.

25. Eggenreich, B. et al. (2020), Journal of Biotechnology 324S, 100022.

26. Brinson, R. G. et al. (2019), mAbs, 11, 94-105.

27. WO 2021/148411

28. WO 2014111458

29. McAlindon E, et al. (2015); Heart; 101; 155-160.

30. Kramer CM, et al. (2020); J Cardiovasc Magn Reson; 22; 17

31. Ware J E (1993), SF-36 Health Survey: Manual and Interpretation Guide.

32. Ware J E (2000), SF-36 health survey update. Spine; 25 (24); 3130-3139.

33. Bernklev T et al. (2005), Inflamm Bowel Dis; 11 (10); 909-918

34. Feagan B G et al. (2007), Am J Gastroenterol; 102; 794-802

35. Feagan B G et al. (2017), Aliment Pharmacol Ther; 45; 264-275

36. Shinde A V et al. (2015), Circ Res; 117:10-1

37. Zhao et al. (2020), Cell Mol Med.; 24:1189-1199

38. Korf-Klingebiel, M et al. (2021), Circulation, 144 (15), 1227-1240

39. Ibanez, B et al. (2019), J Am Coll Cardiol, 74 (2): 238-256.

40. Gibson and Schomig (2004), Circulation, 109:3096-3105.

41. Martin and Rankin, Heart 1999;82:163-16

42. WO 2023/233034 A1

1. A pharmaceutical formulation comprising:
(a) 0.5 mg/ml to 200 mg/ml of a myeloid-derived growth factor (MYDGF) protein;
(b) 10 mM to 100 mM of a buffer;
(c) 1 mM to 50 mM of a stabilizer;
(d) 20 mM to 250 mM of a tonicity agent; and
(e) 0.01% to 0.1% (w/v) of a surfactant;
wherein the composition has a pH of 5.0 to 7.0.

2. The pharmaceutical formulation of claim 1, wherein said buffer is selected from the group consisting of an acetate buffer, a citrate buffer, a histidine buffer, a succinate buffer, a phosphate buffer, and a tromethamine buffer.

3. The pharmaceutical formulation of claim 2, wherein said buffer is a histidine buffer.

4. The pharmaceutical formulation of claim 3, wherein said histidine buffer is present in the composition at a concentration of 10 mM to 30 mM.

5. The pharmaceutical formulation of claim 1, wherein said stabilizer is selected from the group consisting of methionine, arginine, glycine, proline, lysine, and cysteine.

6. The pharmaceutical formulation of claim 5, wherein said stabilizer is methionine.

7. The pharmaceutical formulation of claim 6, wherein said methionine is present in the composition at a concentration of 5 mM to 15 mM.

8. The pharmaceutical formulation of claim 1, wherein said tonicity agent is selected from the group consisting of sucrose, trehalose, sorbitol, mannitol, and dextrose.

9. The pharmaceutical formulation of claim 8, wherein said tonicity agent is sucrose.

10. The pharmaceutical formulation of claim 3, wherein said sucrose is present in the composition at a concentration of 100 mM to 250 mM.

11. The pharmaceutical formulation of claim 1, wherein said surfactant is selected from the group consisting of polysorbate 20, polysorbate 80, and poloxamer 188.

12. The pharmaceutical formulation of claim 11, wherein said surfactant is polysorbate 80 or polysorbate 20.

13. The pharmaceutical formulation of claim 12, wherein said polysorbate 20 is present in the composition at 0.02 to 0.06% (w/v).

14. The pharmaceutical formulation of claim 1, wherein said MYDGF protein comprises or consist of an amino acid sequence selected from the group consisting of SEQ ID NOs: 1, 2, 3, 6, 11 or 13.

15. The pharmaceutical formulation of claim 14, wherein said MYDGF protein comprises or consist of the amino acid sequence of SEQ ID NOs: 1.

16. The pharmaceutical formulation of claim 1, wherein said MYDGF protein is present in the composition at a concentration of 1 mg/ml to 100 mg/ml.

17. The pharmaceutical formulation of claim 16, wherein said MYDGF protein is present in the composition at a concentration of 40 mg/ml to 60 mg/ml, and preferably, 50 mg/ml.

18. The pharmaceutical formulation of claim 1, wherein the pH of the formulation is 5.2 to 6.2.

19. The pharmaceutical formulation of claim 18, wherein said wherein the pH of the formulation is 5.4 to 6.0.

20. The pharmaceutical formulation of claim 1, comprising or consisting of:
(a) 0.5 mg/ml to 200 mg/ml of a myeloid-derived growth factor (MYDGF) protein;
(b) 10 mM to 100 mM of a histidine buffer;
(c) 1 mM to 50 mM methionine;
(d) 20 mM to 250 mM sucrose; and
(e) 0.01% to 0.1% (w/v) polysorbate 20 or polysorbate 80;
wherein the composition has a pH of 5.0 to 7.0.

21. The pharmaceutical formulation of claim 1, comprising or consisting of:
(a) 40 mg/ml to 60 mg/ml of a myeloid-derived growth factor (MYDGF) protein;
(b) 10 mM to 30 mM of a histidine buffer;
(c) 5 mM to 15 mM methionine;
(d) 100 mM to 250 mM sucrose; and
(e) 0.02% to 0.06% (w/v) polysorbate 20 or polysorbate 80;
wherein the composition has a pH of 5.0 to 6.5.

22. The pharmaceutical formulation of claim 1, comprising or consisting of:
(a) 45 mg/ml to 55 mg/ml of a myeloid-derived growth factor (MYDGF) protein;
(b) 18 mM to 22 mM of a histidine buffer;
(c) 9 mM to 11 mM methionine;
(d) 200 mM to 240 mM sucrose; and
(e) 0.02% to 0.06% (w/v) polysorbate 20 or polysorbate 80;
wherein the composition has a pH of 5.7+0.3.

23. The pharmaceutical formulation of claim 1, comprising or consisting of:
(a) 50 mg/ml of a myeloid-derived growth factor (MYDGF) protein;
(b) 20 mM of a histidine buffer;
(c) 10 mM methionine;
(d) 220 mM sucrose; and
(e) 0.04% (w/v) polysorbate 20 or polysorbate 80;
wherein the composition has a pH of 5.7+0.3.

24. The pharmaceutical formulation of claim 1, comprising or consisting of:
(a) 50 mg/ml of a myeloid-derived growth factor (MYDGF) protein which comprises the amino acid sequence of SEQ ID NO:4.
(b) 20 mM of a histidine buffer;
(c) 10 mM methionine;
(d) 220 mM sucrose; and
(e) 0.04% (w/v) polysorbate 20 or polysorbate 80;
wherein the composition has a pH of 5.7+0.3.

25. The pharmaceutical formulation of claim 1, comprising or consisting of:
(a) 50 mg/ml of a myeloid-derived growth factor (MYDGF) protein which comprises or consists the amino acid sequence of SEQ ID NO:1.
(b) 20 mM of a histidine buffer;
(c) 10 mM methionine;
(d) 220 mM sucrose; and
(e) 0.04% (w/v) polysorbate 20;
wherein the composition has a pH of 5.7+0.3.

26. The pharmaceutical formulation of claim 1, comprising or consisting of:
(a) 50 mg/ml of a myeloid-derived growth factor (MYDGF) protein which comprises or consists the amino acid sequence of SEQ ID NO:1.
(b) 20 mM of a histidine buffer;
(c) 10 mM methionine;
(d) 220 mM sucrose; and
(e) 0.04% (w/v) polysorbate 80;
wherein the composition has a pH of 5.7+0.3.

27. The pharmaceutical formulation of claim 1, wherein said formulation is in a liquid form or in a lyophilized form.

28. The pharmaceutical formulation according to claim 27, wherein said formulation is in a liquid form.

29. The pharmaceutical formulation of claim 1, wherein said formulation is formulated as a medicine.

30. A method of treating a myocardial infarction in a subject, preferably a human subject, the method comprising administering a pharmaceutical formulation according to claim 1 to a subject in need thereof.

31. The method of claim 30, wherein said myocardial infarction is a ST-segment elevation myocardial infarction (STEMI).

32. The method of claim 30, wherein said administering step comprises administering said pharmaceutical formulation to said subject by intravenous infusion.

33. The method of claim 32, wherein said method further comprises subjecting said subject to percutaneous coronary intervention (PCI) to restore blood flow to heart tissue of said subject.


SEQUENCE LISTING (18 sequences)

SEQ ID NO: 1 - protein, 143 aa, synthetic construct
  REGION 1..143: note = Recombinant MYDGF variant +A
AVSEPTTVAFDVRPGGVVHSFSHNVGPGDKYTCMFTYASQGGTNEQWQMSLGTSEDHQHFTCTIWRPQGKSYLYFTQFKAEVRGAEIEYAMAYSKAAFERESDVPLKTEEFEVTKTAVAHRPGAFKAELSKLVIVAKASRTEL

SEQ ID NO: 2 - protein, 143 aa, synthetic construct
  REGION 1..143: note = Recombinant MYDGF variant +S
SVSEPTTVAFDVRPGGVVHSFSHNVGPGDKYTCMFTYASQGGTNEQWQMSLGTSEDHQHFTCTIWRPQGKSYLYFTQFKAEVRGAEIEYAMAYSKAAFERESDVPLKTEEFEVTKTAVAHRPGAFKAELSKLVIVAKASRTEL

SEQ ID NO: 3 - protein, 143 aa, synthetic construct
  REGION 1..143: note = Recombinant MYDGF variant +G
GVSEPTTVAFDVRPGGVVHSFSHNVGPGDKYTCMFTYASQGGTNEQWQMSLGTSEDHQHFTCTIWRPQGKSYLYFTQFKAEVRGAEIEYAMAYSKAAFERESDVPLKTEEFEVTKTAVAHRPGAFKAELSKLVIVAKASRTEL

SEQ ID NO: 4 - protein, 141 aa, synthetic construct
  REGION 1..141: note = Recombinant MYDGF variant -V
SEPTTVAFDVRPGGVVHSFSHNVGPGDKYTCMFTYASQGGTNEQWQMSLGTSEDHQHFTCTIWRPQGKSYLYFTQFKAEVRGAEIEYAMAYSKAAFERESDVPLKTEEFEVTKTAVAHRPGAFKAELSKLVIVAKASRTEL

SEQ ID NO: 5 - protein, 173 aa, Homo sapiens
  REGION 1..173: note = MYDGF with signal peptide
MAAPSGGWNGVGASLWAALLLGAVALRPAEAVSEPTTVAFDVRPGGVVHSFSHNVGPGDKYTCMFTYASQGGTNEQWQMSLGTSEDHQHFTCTIWRPQGKSYLYFTQFKAEVRGAEIEYAMAYSKAAFERESDVPLKTEEFEVTKTAVAHRPGAFKAELSKLVIVAKASRTEL

SEQ ID NO: 6 - protein, 142 aa, Homo sapiens
  REGION 1..142: note = Mature MYDGF after processing
VSEPTTVAFDVRPGGVVHSFSHNVGPGDKYTCMFTYASQGGTNEQWQMSLGTSEDHQHFTCTIWRPQGKSYLYFTQFKAEVRGAEIEYAMAYSKAAFERESDVPLKTEEFEVTKTAVAHRPGAFKAELSKLVIVAKASRTEL

SEQ ID NO: 7 - DNA, 4975 bp, synthetic construct
  misc_feature 1..4975: note = Expression vector encoding recombinant MYDGF variant +A
  source 1..4975: mol_type = other DNA
agatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagcggtacccgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctctgagcccgaagtggcgagcccgatcttccccatcggtgatgtcggcgatataggcgccagcaaccgcacctgtggcgccggtgatgccggccacgatgcgtccggcgtagaggatcgagatcgatctcatgatcccgtcgacgaaattaatacgactcactataggggaattgtgagcggataacaattcccctctacaaataattttgtttaactttaagaaggagatatacatatggcagtttctgaacctacgacggttgcttttgatgttcgcccgggtggtgttgtacacagctttagccacaacgtcggtccgggcgacaagtatacgtgcatgttcacgtacgcaagccagggcggcaccaatgaacaatggcagatgagcctgggcacttccgaggatcatcaacactttacctgtaccatctggcgtccgcaaggtaagtcgtacctgtacttcacccagttcaaagccgaagtgcgcggtgcggaaatcgagtatgccatggcgtatagcaaagccgcgttcgagcgtgagagcgacgtgccattgaaaaccgaagaatttgaagtgaccaagaccgcagtggcgcatcgtccgggtgcgttcaaagctgagctgagcaaactggtcattgtcgcgaaggcaagccgtaccgagctgtaataggaattcgctagtctagactcgagatcactagtaccggtccatggcagcccgggtaaagcaccgcaatgatggagaattagatccggctgctaacaaagcccgaaaggaagctgagttggctgctgccaccgctgagcaataactagcataaccccgcggggcctcttcgggggtctcgcggggttttttgctgaaagagcttcaaataaaacgaaaggctcagtcgaaagactgggcctttcgttttatctgttgtttgtcgctgcgccgcactcgcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggcaggtgggatccattgaaatacaggaacgcacgctggatggcccttcgctgggatggtgaaaccatgaaaaatggcagcttcagtggattaagtgggggtaatgtggcctgtaccctctggttgcataggtattcatacggttaaaatttatcaggcgcgatcgcggcagtttttcgggtggtttgttgccatttttacctgtctgctgccgtgatcgcgctgaacgcgttttagcggtgcgtacaattaagggattatggtaaatccacttactgtctgccctcgtagccataagcttcaggtggcacttttcggggaaatgtgcgcggaacccctatttgtttatttttctaaatacattcaaatatgtatccgctcgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttaccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgtagatctacatgaccaaaatcccttaacgtgagttttcgttccactgagcgtcagaccccgtagaaa

SEQ ID NO: 8 - DNA, 4975 bp, synthetic construct
  misc_feature 1..4975: note = Expression vector encoding recombinant MYDGF variant +S
  source 1..4975: mol_type = other DNA
agatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagcggtacccgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctctgagcccgaagtggcgagcccgatcttccccatcggtgatgtcggcgatataggcgccagcaaccgcacctgtggcgccggtgatgccggccacgatgcgtccggcgtagaggatcgagatcgatctcatgatcccgtcgacgaaattaatacgactcactataggggaattgtgagcggataacaattcccctctacaaataattttgtttaactttaagaaggagatatacatatgagcgtttctgaacctacgacggttgcttttgatgttcgcccgggtggtgttgtacacagctttagccacaacgtcggtccgggcgacaagtatacgtgcatgttcacgtacgcaagccagggcggcaccaatgaacaatggcagatgagcctgggcacttccgaggatcatcaacactttacctgtaccatctggcgtccgcaaggtaagtcgtacctgtacttcacccagttcaaagccgaagtgcgcggtgcggaaatcgagtatgccatggcgtatagcaaagccgcgttcgagcgtgagagcgacgtgccattgaaaaccgaagaatttgaagtgaccaagaccgcagtggcgcatcgtccgggtgcgttcaaagctgagctgagcaaactggtcattgtcgcgaaggcaagccgtaccgagctgtaataggaattcgctagtctagactcgagatcactagtaccggtccatggcagcccgggtaaagcaccgcaatgatggagaattagatccggctgctaacaaagcccgaaaggaagctgagttggctgctgccaccgctgagcaataactagcataaccccgcggggcctcttcgggggtctcgcggggttttttgctgaaagagcttcaaataaaacgaaaggctcagtcgaaagactgggcctttcgttttatctgttgtttgtcgctgcgccgcactcgcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggcaggtgggatccattgaaatacaggaacgcacgctggatggcccttcgctgggatggtgaaaccatgaaaaatggcagcttcagtggattaagtgggggtaatgtggcctgtaccctctggttgcataggtattcatacggttaaaatttatcaggcgcgatcgcggcagtttttcgggtggtttgttgccatttttacctgtctgctgccgtgatcgcgctgaacgcgttttagcggtgcgtacaattaagggattatggtaaatccacttactgtctgccctcgtagccataagcttcaggtggcacttttcggggaaatgtgcgcggaacccctatttgtttatttttctaaatacattcaaatatgtatccgctcgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttaccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgtagatctacatgaccaaaatcccttaacgtgagttttcgttccactgagcgtcagaccccgtagaaa

SEQ ID NO: 9 - DNA, 4975 bp, synthetic construct
  misc_feature 1..4975: note = Expression vector encoding recombinant MYDGF variant +G
  source 1..4975: mol_type = other DNA
agatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagcggtacccgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctctgagcccgaagtggcgagcccgatcttccccatcggtgatgtcggcgatataggcgccagcaaccgcacctgtggcgccggtgatgccggccacgatgcgtccggcgtagaggatcgagatcgatctcatgatcccgtcgacgaaattaatacgactcactataggggaattgtgagcggataacaattcccctctacaaataattttgtttaactttaagaaggagatatacatatgggtgtttctgaacctacgacggttgcttttgatgttcgcccgggtggtgttgtacacagctttagccacaacgtcggtccgggcgacaagtatacgtgcatgttcacgtacgcaagccagggcggcaccaatgaacaatggcagatgagcctgggcacttccgaggatcatcaacactttacctgtaccatctggcgtccgcaaggtaagtcgtacctgtacttcacccagttcaaagccgaagtgcgcggtgcggaaatcgagtatgccatggcgtatagcaaagccgcgttcgagcgtgagagcgacgtgccattgaaaaccgaagaatttgaagtgaccaagaccgcagtggcgcatcgtccgggtgcgttcaaagctgagctgagcaaactggtcattgtcgcgaaggcaagccgtaccgagctgtaataggaattcgctagtctagactcgagatcactagtaccggtccatggcagcccgggtaaagcaccgcaatgatggagaattagatccggctgctaacaaagcccgaaaggaagctgagttggctgctgccaccgctgagcaataactagcataaccccgcggggcctcttcgggggtctcgcggggttttttgctgaaagagcttcaaataaaacgaaaggctcagtcgaaagactgggcctttcgttttatctgttgtttgtcgctgcgccgcactcgcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggcaggtgggatccattgaaatacaggaacgcacgctggatggcccttcgctgggatggtgaaaccatgaaaaatggcagcttcagtggattaagtgggggtaatgtggcctgtaccctctggttgcataggtattcatacggttaaaatttatcaggcgcgatcgcggcagtttttcgggtggtttgttgccatttttacctgtctgctgccgtgatcgcgctgaacgcgttttagcggtgcgtacaattaagggattatggtaaatccacttactgtctgccctcgtagccataagcttcaggtggcacttttcggggaaatgtgcgcggaacccctatttgtttatttttctaaatacattcaaatatgtatccgctcgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttaccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgtagatctacatgaccaaaatcccttaacgtgagttttcgttccactgagcgtcagaccccgtagaaa

SEQ ID NO: 10 - DNA, 4969 bp, synthetic construct
  misc_feature 1..4969: note = Expression vector encoding recombinant MYDGF variant -V
  source 1..4969: mol_type = other DNA
agatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagcggtacccgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctctgagcccgaagtggcgagcccgatcttccccatcggtgatgtcggcgatataggcgccagcaaccgcacctgtggcgccggtgatgccggccacgatgcgtccggcgtagaggatcgagatcgatctcatgatcccgtcgacgaaattaatacgactcactataggggaattgtgagcggataacaattcccctctacaaataattttgtttaactttaagaaggagatatacatatgtctgaacctacgacggttgcttttgatgttcgcccgggtggtgttgtacacagctttagccacaacgtcggtccgggcgacaagtatacgtgcatgttcacgtacgcaagccagggcggcaccaatgaacaatggcagatgagcctgggcacttccgaggatcatcaacactttacctgtaccatctggcgtccgcaaggtaagtcgtacctgtacttcacccagttcaaagccgaagtgcgcggtgcggaaatcgagtatgccatggcgtatagcaaagccgcgttcgagcgtgagagcgacgtgccattgaaaaccgaagaatttgaagtgaccaagaccgcagtggcgcatcgtccgggtgcgttcaaagctgagctgagcaaactggtcattgtcgcgaaggcaagccgtaccgagctgtaataggaattcgctagtctagactcgagatcactagtaccggtccatggcagcccgggtaaagcaccgcaatgatggagaattagatccggctgctaacaaagcccgaaaggaagctgagttggctgctgccaccgctgagcaataactagcataaccccgcggggcctcttcgggggtctcgcggggttttttgctgaaagagcttcaaataaaacgaaaggctcagtcgaaagactgggcctttcgttttatctgttgtttgtcgctgcgccgcactcgcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggcaggtgggatccattgaaatacaggaacgcacgctggatggcccttcgctgggatggtgaaaccatgaaaaatggcagcttcagtggattaagtgggggtaatgtggcctgtaccctctggttgcataggtattcatacggttaaaatttatcaggcgcgatcgcggcagtttttcgggtggtttgttgccatttttacctgtctgctgccgtgatcgcgctgaacgcgttttagcggtgcgtacaattaagggattatggtaaatccacttactgtctgccctcgtagccataagcttcaggtggcacttttcggggaaatgtgcgcggaacccctatttgtttatttttctaaatacattcaaatatgtatccgctcgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttaccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgtagatctacatgaccaaaatcccttaacgtgagttttcgttccactgagcgtcagaccccgtagaaa

SEQ ID NO: 11 - protein, 143 aa, synthetic construct; identical to SEQ ID NO: 3

SEQ ID NO: 12 - protein, 177 aa, synthetic construct
MGWSLILLFLVAVATRVLSHHHHHHAGSENLYFQGVSEPTTVAFDVRPGGVVHSFSHNVGPGDKYTCMFTYASQGGTNEQWQMSLGTSEDHQHFTCTIWRPQGKSYLYFTQFKAEVRGAEIEYAMAYSKAAFERESDVPLKTEEFEVTKTAVAHRPGAFKAELSKLVIVAKASRTEL

SEQ ID NO: 13 - protein, 148 aa, synthetic construct
VSEPTTVAFDVRPGGVVHSFSHNVGPGDKYTCMFTYASQGGTNEQWQMSLGTSEDHQHFTCTIWRPQGKSYLYFTQFKAEVRGAEIEYAMAYSKAAFERESDVPLKTEEFEVTKTAVAHRPGAFKAELSKLVIVAKASRTELHHHHHH

SEQ ID NO: 14 - protein, 167 aa, synthetic construct
MGWSLILLFLVAVATRVLSVSEPTTVAFDVRPGGVVHSFSHNVGPGDKYTCMFTYASQGGTNEQWQMSLGTSEDHQHFTCTIWRPQGKSYLYFTQFKAEVRGAEIEYAMAYSKAAFERESDVPLKTEEFEVTKTAVAHRPGAFKAELSKLVIVAKASRTELHHHHHH

SEQ ID NO: 15 - protein, 152 aa, synthetic construct
VSEPTTVPFDVRPGGVVHSFSQDVGPGNKFTCTFTYASQGGTNEQWQMSLGTSEDSQHFTCTIWRPQGKSYLYFTQFKAELRGAEIEYAMAYSKAAFERESDVPLKSEEFEVTKTAVSHRPGAFKAELSKLVIVAKAARSELLEHHHHHHHH

SEQ ID NO: 16 - protein, 177 aa, synthetic construct
MGAAPSGGFWTAVVLAAAALKLAAAVSEPTTVPFDVRPGGVVHSFSQDVGPGNKFTCTFTYASQGGTNEQWQMSLGTSEDSQHFTCTIWRPQGKSYLYFTQFKAELRGAEIEYAMAYSKAAFERESDVPLKSEEFEVTKTAVSHRPGAFKAELSKLVIVAKAARSELLEHHHHHHHH

SEQ ID NO: 17 - protein, 148 aa, synthetic construct
VSEPTTVAFDVRPGGVVHSFSQNVGPGNKYTCVFTYASQGGTNEKWQMSLGTSEDHQHFTCTIWRPQGKSYLYFTQFKAEVRGAEIEYGMAYSKAAFERESEVPLKSEEFEVTKTAVSHRPGAFKAELSKLVIVAKVTRSELHHHHHH

SEQ ID NO: 18 - protein, 167 aa, synthetic construct
MGWSLILLFLVAVATRVLSVSEPTTVAFDVRPGGVVHSFSQNVGPGNKYTCVFTYASQGGTNEKWQMSLGTSEDHQHFTCTIWRPQGKSYLYFTQFKAEVRGAEIEYGMAYSKAAFERESEVPLKSEEFEVTKTAVSHRPGAFKAELSKLVIVAKVTRSELHHHHHH